(19) United States
(12) Patent Application Publication
(10) Pub. No.: US 2026/0260766 A1
(43) Pub. Date: Sep. 3, 2026

(54) System and Method for Aggregating Clinical Data, Medical Device Data, and Patient Data for Patient Care and Ongoing Device Performance Analysis

(71) Applicants: Joseph L. Ortiz, Knoxville, TN (US); Keith D. Williams, Germantown, TN (US)

(72) Inventors: Joseph L. Ortiz, Knoxville, TN (US); Keith D. Williams, Germantown, TN (US)

(21) Appl. No.: 19/066,326

(22) Filed: Feb. 28, 2025

(51) Int. Cl. G16H 50/70; G06F 16/11; G06F 16/27; G16H 10/60; G16H 40/67

(52) U.S. Cl. CPC G16H 50/70; G06F 16/116; G06F 16/27; G16H 10/60; G16H 40/67

(57) ABSTRACT

A medical information system captures medical device information and patient information to enable real-time determinations of their safety and appropriateness for use, as well as longitudinal tracking of each medical device implanted or used in specific patients during a specific patient care encounter. The system further makes this same information available to care providers at a later time on an individual as-needed basis to provide care for that patient. The system also receives post-implant recall and other device data, patient biometric data to improve clinical data on the device performance. The data analytics can be applied to both individual patients and selected populations. The information can be made available for review to various interested third parties for various purposes.

### BACKGROUND OF THE INVENTION

### Field of the Invention

The invention pertains to healthcare information systems and methods for capturing commercial status, product specifications, and the required Unique Device Identifier (UDI) of a medical product with extensive device information and combining this information with individual patient demographics and patient encounter-specific information and, more particularly, to systems and methods for identifying and following a medical device and a specific patient before, during, and after a patient procedure.

### Description of Related Art

Currently, neither health care providers, manufacturers, payors, researchers, nor other interested parties have a unified, available system or method to identify and track patients with implanted medical device products, in the US or globally that enables one to determine where, when, or in whom a specific medical device was used. Without this information, it is not possible to identify and track the device to allow optimal patient treatment decisions or determine post-implant device performance characteristics.

Implanted medical devices include those critical to maintaining the patient's life, sustaining or improving the patient's physical function, or alleviating pain for the patient. More broadly, this includes all FDA-regulated medical devices in the United States and regulated medical devices under non-US authorities. The FDA defines all medical devices as Class I, II, or III based on the medical device's impact on human life.

The lack of a comprehensive system for identifying and tracking the products in patients also means there is no way to determine medical device effectiveness without developing a specific patient disease registry or similar data collection plan. Such tracking requires collecting data from the point of care during which a medical device is used for a single patient through to when the medical device is replaced due to failure or complication in that patient, the patient dies, or the device is recalled or discontinued from the market. Such a comprehensive data set would need to include data derived from the clinical encounter record created at the time of the procedure, any follow-on encounter that is tied to the implanted medical device, any subsequent recall or adverse event report for the device, or any biometric data generated from the medical device itself or from patient-provided biometric sensors capturing data directly or indirectly tied to the medical device performance and other potential data sources. It must be understood that the data may come from anywhere across the entire healthcare ecosystem, independent of individual healthcare providers, facilities, manufacturers, or the healthcare clinical systems used by the facility where the original procedure was done. Furthermore, the data set would need to be independent of the format or type of electronic health record or the source, type, or format of the additionally collected patient and device information, including biometric data. There is, at present, no way that all this data can be collected in a way that enables it to be used by any interested parties. Potentially interested parties would include the patient, specific patient health care providers, healthcare systems, payors, researchers, and what are regulators, to name a few.

This healthcare system failure exposes patients worldwide to risks from implanted devices later determined to be counterfeit, implanted after expiration, inappropriate for the procedure based on laterality or other patient factors, deficient or defective, and recalled or associated with adverse events. Furthermore, this lack of information hinders earlier detection of safety signals related to a specific device.

This failure also limits the ability of manufacturers or regulators to conduct adequate surveillance of medical devices. While specific implanted medical devices are becoming network-aware, enabling them to connect over a private network to a centralized monitoring hub, these solutions are one-off solutions that don't provide a generalized solution for device effectiveness and performance analytics, independent of the type of device. Also, these systems cannot integrate patient-generated biometric data that may provide additional insights into the operation or performance of implanted medical devices.

In 2013, the Food and Drug Administration (FDA) issued regulations requiring a human-readable and a machine-readable label called the FDA Unique Device Identifier (UDI) to be created and applied to the label of every manufactured medical device, or to be marked on the device itself. The FDA oversees this regulatory process. The regulation also established the Global Unique Device Identifier Database (GUDID) to aggregate data for every device (Class I, II, and III) based on the created UDI. Furthermore, the UDI for a product shall contain a DI (device identifier) component that specifies the product and a PI (production identifier) component that specifies information such as lot/batch number, expiration date, serial number, a distinct identification number/code for cellular products regulated as a device as a few examples.

The European Union (EU) has a structured regulatory framework for medical devices, which incorporates Unique Device Identification (UDI) as a core component for traceability, safety, and post-market surveillance. Unlike the U.S. FDA's GUDID (Global Unique Device Identification Database), the EU UDI system is part of the broader EUDAMED (European Database on Medical Devices). UDI is required for conformity assessment to ensure traceability during device approvals. The failure in the system tracking medical devices is a global issue, and the invention detailed here addresses all potential medical device databases that may originate outside the United States. The FDA GUDID is widely being adopted, but any specific changes in data format describing the medical device and any additional databases with related information are addressed in the invention via the correlator platform.

The FDA publishes the GUDID, which is publicly accessible. The GUDID has become the reference standard for medical device identification and tracking globally, with variations in the European Union and other developed nations. From the implementation dates included in the regulation, all three medical device classes must have a UDI label or direct part marking with the UDI. The FDA also publishes separate databases with information about medical devices, including the FDA Recall Database and the FDA Adverse Events Database.

While all medical device manufacturers have labeled medical devices with a UDI, and all healthcare information technology (HIT) providers have been required to provide fields to store the data, the end users of the HIT systems are not required to transition to the exclusive use of UDI within their software systems. Technology providers either do not support UDI fully or require a major system update to access the functionality. This inability to fully utilize the UDI system leaves many operational benefits and patient health care benefits unrealized.

While the Centers for Medicare and Medicaid Services (CMS) Office of the National Coordinator mandated that Electronic Health Record systems must be able to record UDI information in the patient record, use of this feature is limited because many users continue to use the manufacturers' item and catalog numbers for product identification. Thus, UDI numbers are not being used consistently to identify products in Inventory and Supply Chain Management Systems, Enterprise Resource Planning (ERP) Systems, Surgical Planning, Laboratory Information Management Systems (LIMS), Operating Room Management Systems (ORMS), Preoperative and Perioperative Information Systems, etc.

While various approaches have been defined to track medical devices in inventory for recalls, these systems do not prospectively track the implantation of the medical device in the patient, nor do they provide a mechanism for capturing and associating medical device data with a specific patient post-implantation.

Recall management in even the largest, most sophisticated hospital systems cannot track patients who move outside their immediate care. Therefore, recall management can often fall short of notification for all patients who have an implanted device that is later recalled. Unfortunately, the FDA Recall database does not include UDI for all entries. The FDA has not yet standardized the information required of manufacturers detailing the product identification for the recall. This makes matching devices for recalls a challenge, and this invention addresses this.

Some very large-scale hospital systems have implemented specific, targeted projects to determine the implementation issues of using UDI to identify and track medical devices and device usage up to the time of implantation with selected patient populations or by specific providers. While these hospital systems may have saved the medical device implant data in their electronic health record system, it is not available outside the index system to other individual health care providers or providers within organizations that may now be treating a patient who previously had an implant done at the large hospital system. Also, the recorded implant data is not prospectively monitored for recall, safety signals, or performance in the patient.

Even for those healthcare systems that support UDI capture for pre-procedure and during the procedure, there are no solutions readily available for tracking UDI and other device information specific to a patient to improve the patient's future care or track the medical device performance for that patient in the future. Consequently, no system can improve patient care or track cohorts of patients with the same comorbidities at various times after their procedures.

No general-purpose system exists that can incorporate UDI medical product identification and device tracking pre-, during-, and post-procedure and do so in a way that associates the medical products and device data with the patient healthcare data to improve long-term patient care quality and safety. Further, there is no general-purpose system existing that additionally identifies and tracks a medical device pre-procedure, when the device is first received as a procured new inventory item to be stocked for future use in a procedure, and that provides notifications of recalls, adverse events, or expiration changes to those items sitting in inventory waiting to be used. There is no solution available for small, medium, and larger-sized hospitals to increase UDI adoption without the same significant investments made by large hospitals to create one-off solutions.

The lack of correct and accessible patient medical device implant records that begin at the point of the implantation procedure and continue over time, independent of the hospital provider system, and are linked to the patient seeking care, has been, and remains a significant gap in patient care since the beginning of modern medicine.

The Food and Drug Administration continues making significant investments to create a more robust medical device surveillance system that can identify “safety signals” earlier in the life of a medical device, post-market. These efforts rely on anonymized patient data and would not enable sophisticated data analytics, machine learning, and device effectiveness reporting. None of these efforts address how to begin the capture of surveillance data from the procedure point of care during which a specific patient received one or more specific, identifiable medical devices.

Funded clinical trials have been conducted in specific disease areas, enabling a longitudinal record for patient care or medical outcome analytics. Further, systems today rely on post-market surveillance data, which almost always lacks details of the patient's specific medical history or comorbidities related to the device and device performance.

For all healthcare systems that currently do electronically read UDI labels and look the information up against the FDA GUDID Medical Device Database, several intractable problems persist. First, the predominant current solution for barcode scanning is a laser-based barcode reader. This device can read a barcode and provide the data read. Still, these systems cannot tell the user if the barcode laser-scanned is a UDI label or directly tell the user the fitness for the use of that medical device based on the UDI label data. Systems that scan for a UDI barcode and find the UDI data typically insert the recognized data into a field within the proprietary software solution. They do not perform a real-time validation of the device for recalls, adverse events, expiration, or other critical correctness for use device concerns such as “contains latex,” “MRI compatible,” “Sterilization Method,” or other critical related medical device product information. In these existing types of systems, the various related other FDA and third-party medical device databases, ontologies, and taxonomies are not generally cross-referenced for the purpose of determining whether or not a medical device is fit to use in a medical procedure (i.e., not expired, recalled, or subject to material adverse event reporting) or the correct part to use for in a procedure (i.e., correct laterality, or no metal allergies, for example). For example, databases for recalls and adverse events and disparate other sources of medical device-specific information, such as from the manufacturer instructions for use, research studies, trade reports, distributor pricing, and so on, are not cross-correlated to provide the health care system single source of complete and accurate truth about the medical device in question.

Clinical registries are a well-known solution for capturing and combining data to support retrospective and prospective analyses of the effectiveness of medical treatments, including devices. Registries in healthcare collect a variety of data sets depending on their purpose-whether for clinical research, post-market surveillance, quality improvement, or public health monitoring. Medical registries have traditionally focused on specific conditions or diseases or medical device types, such as hip osteoarthritis or a total hip replacement, not a specific device. This means a total hip arthroplasty component made by a particular vendor, with specific design characteristics and materials and specific geometries and dimensions, which the UDI of the implant identifies. The specific details of the device and the patient contained within the registry are not available to those not participating. The registry comprises historical data collected and can be used to identify trends, outcomes, and associations for patient response to a particular medical device group. This type of analysis is helpful in understanding historical trends and evaluating the long-term effectiveness and safety of the average device used to treat a specific condition or disease in a group of patients. These retrospective registries can be used for data mining, statistical analysis, comparative studies, or risk assessments based on adverse event reporting. Examples of retrospective registries include the FDA Sentinel Initiative, the National Joint Registry, the International Consortium of Orthopedic Registries, and the American College of Cardiology's National Cardiovascular Data Registry (NCDR).

Clinical registries containing prospective data are now being developed and focus on collecting and analyzing data moving forward from a specific point in time. Prospective registry analysis monitors outcomes as they occur and helps evaluate new treatments or devices in a more timely manner than traditional healthcare records systems. Typically, patient cohort populations are identified and enrolled based on specific criteria. Regularly scheduled follow-ups may be used to collect outcome data. The health care provider usually determines participation in a registry based on a particular set of agreed-upon criteria, not the patient, and many patients may be excluded based on the requirements. Also, the registry information is generally available to the participants, not other health care providers. The funding to create and maintain registries is provided by various public and private entities with individual entity objectives for the data in the registry. Registries typically do not collect complete patient encounter records detailing the diagnosis, treatment, and medical devices used at the point of care. Normally, suppose data is collected for a registry. In that case, the information is sparse and may only include a diagnosis or biopsy result or a medical device used but reported to a specific orthopedic or cardiac registry. No details of the procedure, the provider, the facility, or patient history, procedure notes, or follow-up data are captured in these registries. The invention represents the real-world use of a specific device in any patient and facility. Collecting a complete record of the patient demographics, diagnosis, a complete procedure bill of materials, an implanted device list, details of the facility, and, more importantly, post-procedure clinical notes, instructions to the patient, and so on. Even more important, these registries capture only a single data point concerning the tracking of the disease or device. All that is captured is the disease/diagnosis or the medical device and where it was used. There is no longitudinal tracking of a single patient as it relates to the use of a specific medical device for a particular diagnosis

What is needed, therefore, is a single platform that comprehensively covers any patient in any healthcare system, for any medical device usage for any type of diagnoses and conditions, independent of bodily function or organ system involvement and comorbidities, that is designed to systematically capture medical device and patient information at the implantation procedure point of care and further incorporate future information that arises, such as product recalls and adverse events after the implanted device is in service, and that makes the patient and medical device data available to authorized users via an online data-access portal, granting access according to the user's access rights to patient protected healthcare data, that through automated procedures updates source databases and cross-correlates new data from existing and newly added data sources for the enrichment of the data used for analysis of medical devices and patient procedure medical device effectiveness, and that enables the development of traditional statistical analysis tools (descriptive an analytics, inferential statistics, time-series, analysis), prospective and retrospective analyses (cohort analysis, comparative effectiveness research prospective data collection and analysis), big data/data lake analysis m(aggregating unstructured data, graphic analytics), machine learning and AI-driven analysis (supervised learning (predictive analytics), unsupervised learning (pattern discovery), natural language processing (NLP), reinforcement learning for personalized medicine), real-world evidence (RWE) and epidemiological analysis (post-market surveillance, health economics and outcomes research (HEOR), public health surveillance and adverse event reporting).

### Objects and Advantages

Objects of the invention include: providing a system to identify and track medical devices using FDA mandated UDI labels and markings; providing a system to verify Fitness for Use and Correctness for Use before a device is used for patient care; providing a system that maintains medical device data, patient data, and procedure data in a separate, unified HIPAA-compliant patient procedure and medical device system, allowing the system to be accessed by the patient directly, any health care provider or healthcare organization information system given permission by the patient; providing a system that captures at the time of encounter, the identity of and relevant data about the implanted medical device, patient, clinical provider, facility, and adds this information to a system that can later be used by patients, providers, device manufacturers, regulators, and clinical researchers; providing: 1) a complete enriched data set integrating diverse data sets (structured, unstructured, ontological, taxonomical) identifying and describing all medical devices and medical device impacts on human physiology, providing 2) a more complete data set identifying and defining a specific patient's clinical data, and pairing these two data sets, and providing a method to share the paired data sets with all appropriate entities including patients, providers, researchers, GPOs, regulators, among others; and longitudinally monitoring changes to one or more of the data sets using analytic tools, insights into specific outcomes for patients based on specific characteristics of the patients, and insights into device-specific performance and safety. The shared information can be provided as patient-identified or de-identified as appropriate.

### BRIEF SUMMARY OF THE INVENTION

According to one aspect of the invention, a method for identifying and tracking medical devices comprises the following steps of:
a) acquiring UDI for medical device(s) to be used in a procedure on a selected patient;
b) validating each device for use by checking at least one device database;
c) performing the procedure on the patient;
d) preparing a file recording the complete list of all UDIs for products used during the procedure, patient clinical data, and clinical encounter data as part of the USCDI standard to create a Complete UDI Encounter Record (CUER);
e) adding the file to a database of Complete UDI Encounter Records;
f) updating the file going forward on an as-needed basis as part of the aggregated CUERs;
g) making the file available to various selected parties under selected access rules.

According to another aspect of the invention, a method for identifying and tracking medical devices comprises the following steps of:
a) providing a server device including searchable databases comprising at least one selected from the group consisting of:
   a first database listing commercially available medical devices;
   a second database listing recalled medical devices;
   a third database listing reported adverse events associated with medical devices;
   a fourth database listing commercially available tissue and biologic devices; and,
   a fifth database listing encounter records for medical procedures in which medical devices were used in individual patients and the corresponding UDIs of said devices;
b) providing a plurality of client devices through which users may query said searchable databases and may upload encounter records to said encounter records database;
c) before performing a medical procedure, using a client device to:
   acquire the UDI for each device selected for possible use in said medical procedure;
   query said at least one database(s) and reject any selected device that is expired or subject to recall; and,
   prepare a complete bill of materials for the said procedure;
d) after said medical procedure is completed, using a client device to upload an encounter record to said encounter record database, said encounter record comprising at least:
   date of procedure;
   type of procedure;
   patient identifying information;
   patient clinical information (USCDI)
   provider identifying information;
   physician identifying information; and,
   complete list of all medical devices used and their UDIs; and,
e) making said encounter database accessible at future times to selected users under selected access rules.

### BRIEF DESCRIPTION OF THE DRAWINGS

The drawings accompanying and forming part of this specification are included to depict certain aspects of the invention. A clearer conception of the invention and the components and operation of systems provided with the invention will become more readily apparent by referring to the exemplary, and therefore non-limiting embodiments illustrated in the drawing figures, wherein like numerals (if they occur in more than one view) designate the same elements. The features in the drawings are not necessarily drawn to scale.

FIG. 1 depicts the overall system architecture in accordance with some aspects of the invention.

FIG. 2 is a diagram showing the components of a correlator process in accordance with some aspects of the invention.

FIG. 3 shows the UDI-centric workflow for devices used in one procedure on one patient in accordance with some aspects of the invention.

FIG. 4 is a schematic diagram of a user session interface in accordance with some aspects of the invention.

FIG. 5A-B illustrates the Single Client Service Environment (SCSE), FIG. 5A, and its interconnection with the Patient Medical Device Management Environment, FIG. 5B, in accordance with some aspects of the invention.

FIG. 6 is the system architecture diagram of the patient-encounter-specific medical device tracking system.

FIG. 7 illustrates the CUER Workflow, pre-procedure, in accordance with some aspects of the invention.

FIG. 8 illustrates the CUER workflow during a procedure in accordance with some aspects of the invention.

FIG. 9 illustrates the CUER workflow during a revision procedure in accordance with some aspects of the invention.

FIG. 10 illustrates identifying and associating a Unique Global Patient ID to a patient in accordance with some aspects of the invention.

FIG. 11A-B illustrates the process of combining data collected from separate healthcare delivery organizations in various incompatible data formats in accordance with some aspects of the invention. FIG. 11A depicts a first portion of the process, and FIG. 11B depicts a second portion of the process.

FIG. 12 illustrates the architecture of the End-User Portal, which provides Portal Users with role-specific access to the data contained in the PHCDB 900.

FIG. 13 is a state diagram illustrating the stages that a Patient CUER 910 undergoes from creation to final update with post-procedure data.

FIG. 14 depicts the classes or types of data that will be collected in the patient-specific CUER 910.

FIG. 15 is an expansion of FIG. 11B showing the conversion process from various medical data formats into the SINDF format 1550. Depicted are the different data sources, each with a 1 to 1 mapping to a conversion template 1682.

### DETAILED DESCRIPTION OF THE INVENTION

The invention performs several functions within the general field of healthcare information technology. In one activity, it captures and processes medical device data, in particular the Unique Device Identifier (UDI), preferably by the user of a camera or image sensor-enabled mobile phone or tablet device to capture the image of a UDI contained on a label or directly marked on a medical device; one or more databases are then automatically consulted to determine if a device is fit for use and correct for use or not. Suppose the device is usable and selected for use by a specific patient. In that case, the second activity is to collect patient-specific information and procedure information, including an inventory of all devices used or implanted during the procedure and placing the aggregated information as a Complete UDI Encounter Record into a database. The database is configured such that searching may be done by patient, provider, device UDI, and other fields, as will be detailed in several examples. In a third activity, users may later upload additional information to the record. The system may periodically scan all the records to find patients with devices that have been subject to later recalls or reported adverse events. The system will continue tracking each medical device to its ultimate point of use (or revision of any cause), but more importantly, provide the patient and designated health care providers with a centralized place to access the identity of and information about any devices in his own body. The invention thus enables medical device surveillance that begins at the point of device implantation into a patient and is then ongoing with time. Further, this invention also makes it possible for the user to camera-scan medical devices for fitness at any time in the medical device lifecycle. Examples include medical device distributors maintaining a large inventory of devices for resale who can now track the recall, adverse events, and expirations of products on-shelf. It can be envisioned that the invention can be embodied as a machine vision system in a medical device distribution warehouse or even as a plugin to an existing commercial cargo carrier's tracking system if there is a significant volume of medical device packages transported over time. Also, any healthcare organization that procures medical devices can use the invention for procurement receiving inspection, benefiting from a near real-time acceptance or rejection of the delivery.

The inventive methods rely on a configurable collection of online computation, storage, search, and retrieval services made available to create secure, scalable, fault-tolerant, and customer-friendly data collection and management services subject to rigorous security standards and strict data access controls.

Momentum is increasing for the adoption of the US Food and Drug Administration's Medical Device identification regulation [“Unique Device Identification System,” 21 CFR Part 830, Effective Date Sep. 24, 2013] within the ‘last mile’ of the healthcare delivery system. Tracking purchased products to their specific patient use is needed globally across the healthcare ecosystem. This begins in the shipment receiving area, any inventory rooms, and when prepared and separated for use in a procedure for patient care.

Offering a universal solution for medical device utilization management, based on the FDA UDI regulation, would enable organizations to quickly begin capturing inventory data, verify fitness for use, and track any future changes to any products managed by the system. As an open system platform, the inventive system can connect with third-party inventory management or Enterprise Resource Planning solutions, effectively extending the capabilities of those existing platforms.

The invention also provides a simple and comprehensive solution for collecting longitudinal patient medical device data at the encounter level. It effectively establishes time t=0 for the start of tracking patient impacts, responses to, and benefits from the identified medical device used or implanted during a procedure.

The invention enables patient healthcare data and medical device performance data specific to a single patient to be collected independently of any originating source, e.g., healthcare system, university, medical device manufacturer, medical device distribution firm, or research facility. This means all the data is converted into the invention's internal open systems-based approach for data aggregation, called SINDF, System Internal Normal Data Format, 828, 833, independent of the source data format of these typical classes of healthcare information technology systems: ERP/Supply Chain and Medical Inventory Management Systems, EHR, Hospital Information Systems (HIS) and Health Information Exchange (HIE), Surgical and Perioperative Management Systems, Medical Device Integration and Remote Monitoring Systems, Radiology and Medical Imaging Systems, Radiology and Medical Imaging Systems, CVIS (Cardiovascular Information Systems, Laboratory Information Management Systems (LIMS) and Pathology Systems, Clinical Research and Trial Management Systems, Population Health and Analytics Platforms, Home Healthcare and Telemedicine Systems, Biomedical and IoT Device Data Platforms.

After a procedure or implantation, optimal patient care inherently requires access to the patient encounter record regardless of where the procedure was done or where the patient is being treated now. Access to this type of historical patient procedure data is generally unavailable and is usually confined to a single healthcare system when performing a search.

### EXAMPLE

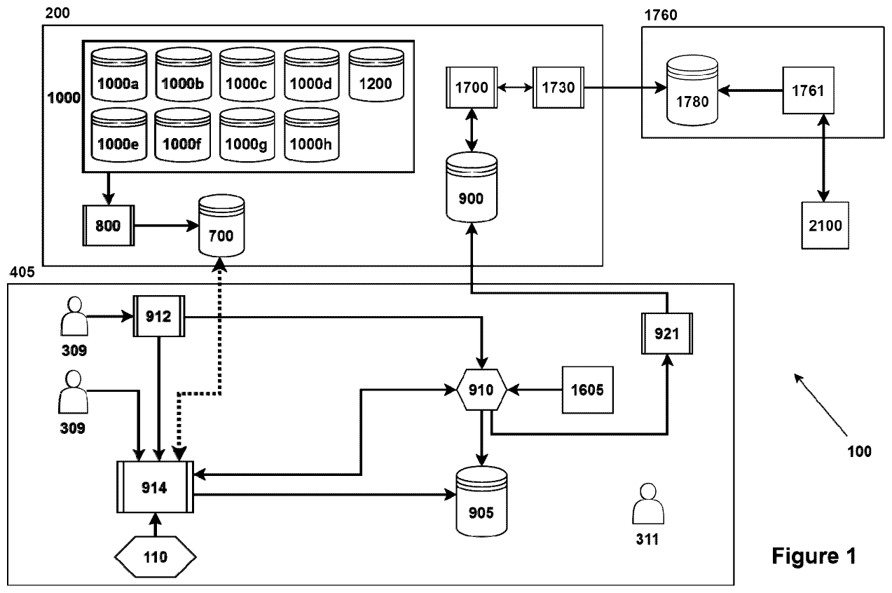

FIG. 1 presents a high-level representation of the general functions of the Invention. The invention covers the areas detailed in 100, such as the Central Server System. The central server system consists of three major subsystems: the Patient Medical Device Management Environment 200; one or more of the Single Client Service Environments 405; and finally, the Patient History of CUER (PHC) Portal Environment 1760. The Patient Medical Device Management Environment 200 is a highly secure online private environment of computational infrastructure capabilities and resources that provide the core backend processing of the invention, including running the Medical Device Correlator Process 800 to create the Medical Device Master Database 700 or for example, generate a recall event notification to be sent to Clients for patient notification. The Single Client Service Environment 405 depicted in the figure is for one client user, and offers each client a private, VLAN-protected web server, database, and processing service specific to that customer's users, locations, and patient population. Within 405, users can camera-scan medical device packaging labels to determine if that individual unit is fit for use 914, they can create a CUER record 910, interface their system-specific healthcare IT vendor systems 1605 for access to data to be added to the CUER 910, and ultimately they can push a “Final” 927 CUER to the Patient History of CUERs Master Database 900, making it available for future use and reference.

In the medical device management environment 200, local mirrors of external data sources 1000 preferably include the OpenFDA GUDID 1000a, OpenFDA Recalls 1000b, etc. The Medical Device Correlator Process 800 converts all information to System Internal Normal Data Format 828 to create and maintain the Medical Device Master Database 700.

In the client service environment 405, at the healthcare delivery organization, a planned medical procedure will involve patient 311 and medical device(s) 110. A clinical user 309 at the provider organization initiates the creation of a new CUER 910 for the scheduled procedure at 912. A technician 312 or another clinical user 309 validates each device by camera-scanning UDI label(s) or marked parts 914. Each item is added to a single client inventory list 917 in the Single Client Database 905. All medical devices, except for what is referred to as “Trunk Stock,” will be scanned into the SCSE 405. Trunk Stock can be camera-scanned and validated 914 in the procedure room, outside the sterile field. These steps are generally done using all existing local HIT solutions (e.g., ERP and EHR systems). The single CUER 910 is pushed to the Patient History of CUERs Master Database (PHCMD) 900, containing such records for all patients and encounters for all clients.

In portal environment 1760, portal data users 2100 access CUERs based on access rights. The PHCMD 900 files may be periodically updated based on updates in the MDMD 700.

### Workflow.

The invention may be most easily understood in terms of the overall workflow, which includes some activities done before a procedure, some during the procedure, and some after the procedure. The general steps in this workflow include the following:
a) acquire UDI for medical device(s) to be used in a procedure on a selected patient;
b) validate each device for use by checking at least one device database;
c) perform the procedure on the patient;
d) prepare a first file recording the complete inventory by UDI of all medical devices implanted in the patient or used during the procedure;
e) prepare a second file containing specific patient data and clinical data contained within the EHR of the healthcare system to create an encounter record;
f) combine the complete inventory UDI file with the encounter record to create the Complete UDI Encounter Record (CUER) file;
g) update the CUER file going forward on an as-needed basis;
h) make the CUER file available to various selected parties under selected access rules.

It will be understood that the various steps in the workflow may be performed in different sequences and by different people, as will become apparent from consideration of the following examples. It will be further understood that many other activities and steps may be performed by various users for various purposes beyond the simple steps outlined above.

When a patient goes to a physician to address a health concern, the physician will make a diagnosis, assigning an ICD10 code to the patient, which will be included in the electronic health record system. The physician 310 or other clinical user 309, will coordinate with the patient to schedule a time for a procedure. The procedure scheduling is performed, assigning the physician at that time, and booking the selected procedure room. The surgical planning system or EHR subsystem creates a procedure record and populates it with the surgeon's preference card of items for the selected procedure. The plan also provides for coordinating and scheduling support staff, anesthesiology, and so on. The preference card pick list is shared with the supply chain/inventory to trigger supply verification and coordinate for pre-procedure cart pull.

### Example

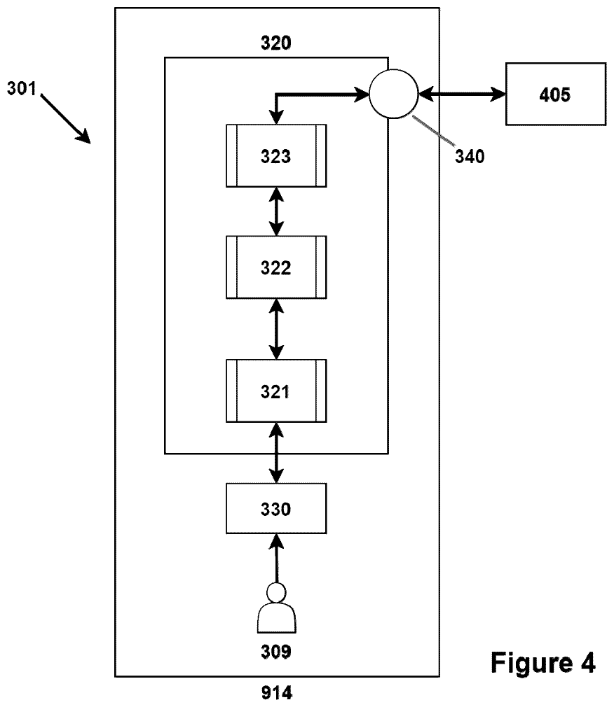

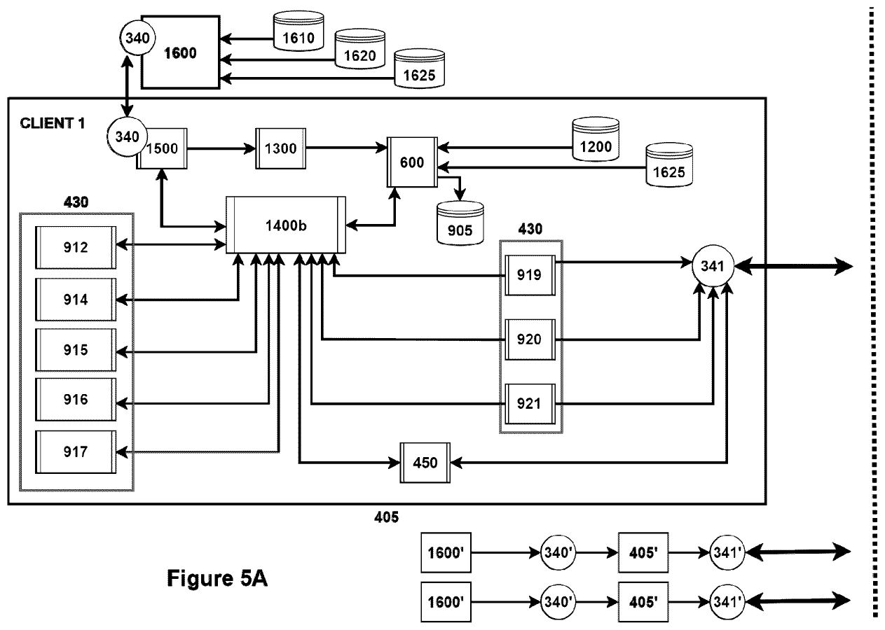

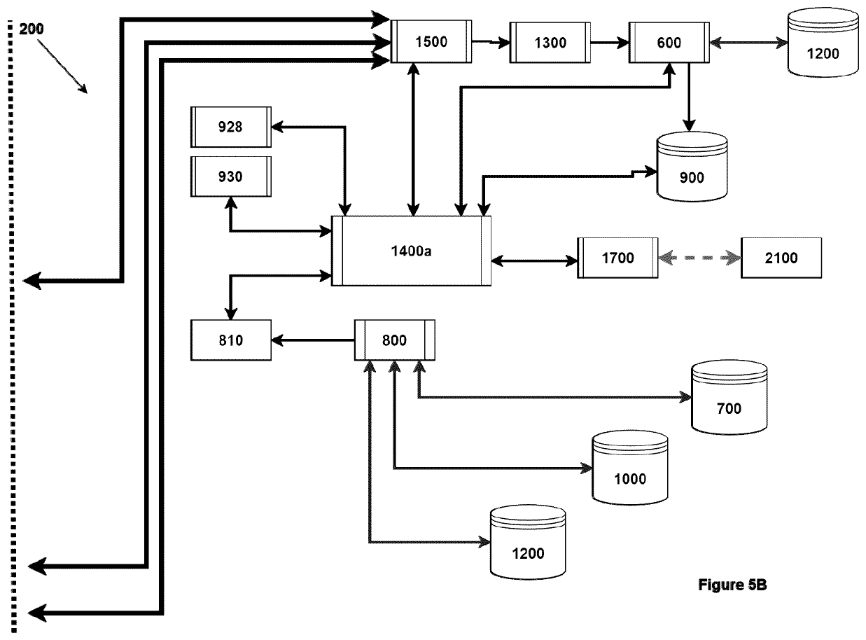

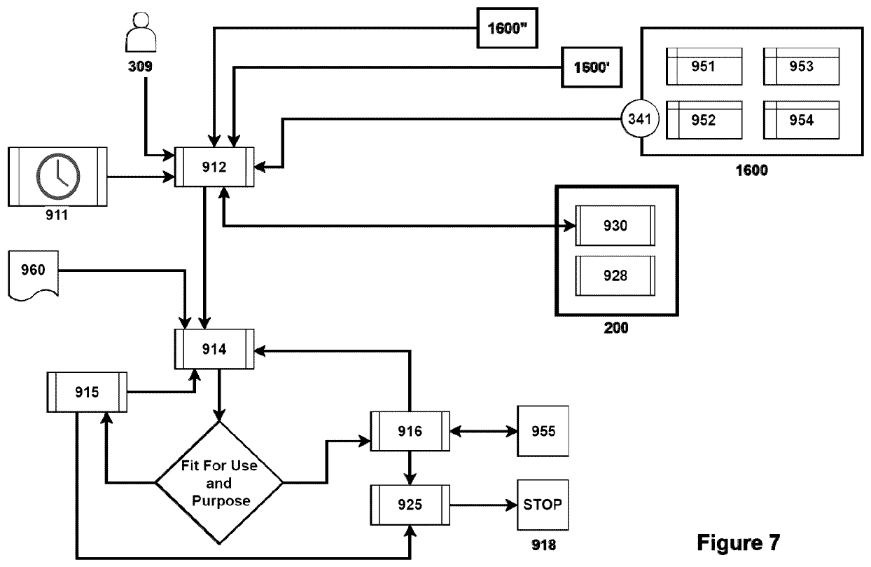

Before the procedure, as shown in FIG. 7, when a new procedure is scheduled, 911, either before the cart pull or at the beginning of the cart pull, a clinical User 309, assigned to pulling the cart, or another manager or administrator initiates creation of the new CUER via 912 for this patient procedure. During the cart pull process, medical devices in need of current verification of fitness for use are taken from inventory 960. The medical device UDI package label is optically scanned, preferably using a mobile device with a camera (e.g., 330 FIG. 4), enabling processing by the cart pull technician using a mobile application comprising an Automatic Identification and Data Capture (AIDC) system. The preferred embodiment of 330 is a mobile device with an image or vision sensor. Alternatively, 330 can be a device that comprises a traditional bar code reader paired with an image or vision sensor that is able to capture a 2D image data set of the UDI device identifier on the device label. The image captured by the camera-scanner will contain a barcode with the medical device UDI information, including the Device Identifier (DI) and the Production Information (expiration, lot/batch, serial no., date of manufacture, and donor ID for biologics). The mobile application device with a camera client queries the dedicated web server (450 in FIG. 5A), which then processes the UDI data from the selected medical device label or part marking to identify the device, check for recalls or adverse events, and verify that the chosen device is fit for use and correct for use based on the scheduled procedure and patient diagnosis. Once all requested items have been scanned and verified, the patient data, procedure data, and inventory of medical devices automatically populate fields in the newly created CUER, whose state is set to be “Initial” and is preloaded with data from connect Healthcare IT vendor systems 1600, 1600, etc. For example, during its creation, the CUER 910 is loaded with patient demographic and specific clinical data and comorbidities that are retrieved from the EHR 1600 and 1625 and populated into the developing CUER 910.

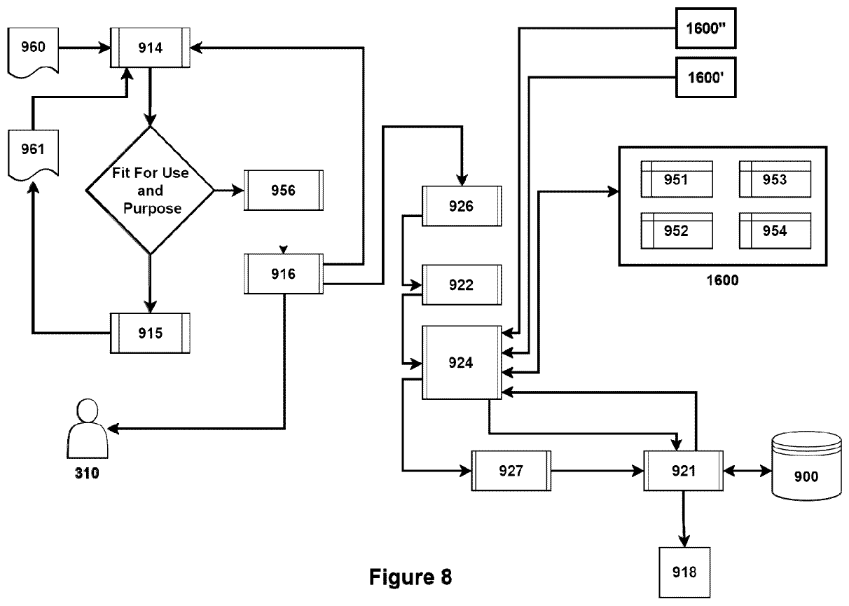

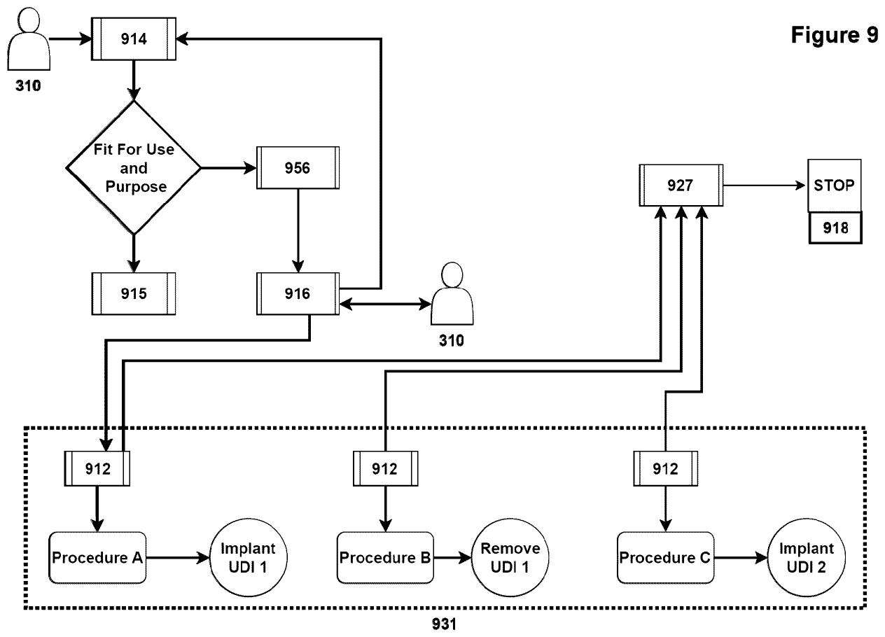

When the procedure is performed, FIGS. 8-9, deviations from the original pick list may occur as additional components or certain pulled items are unnecessary. For example, manufacturer representatives with stocks of available medical devices may be present during procedure 916 and receive requests to provide specific items for use. In this case, outside the operating room sterile field, the representative selects the item and hands it off to staff. The item's medical device identification label is captured for fitness 914 for use processing and correctness for use processing. If fit for use, the system adds it to the inventory bill of materials for procedure 916, and the device can be used. Otherwise, having captured a not-fit-for-use exception, the device can be returned to the representative, and another unit with the same Device Identifier (DI) can be selected. Alternatively, implants may be chosen from trays within the sterile field that has been individually directly marked with UDI data or placed in trays or other organizational, physical methods enabling UDI labeling to be associated with the device, and are scanned within the sterile field, with an identical workflow.

After the procedure is completed, FIG. 8, unused devices are scanned, returned to inventory 922, and not included in the complete UDI encounter record (CUER). The CUER is updated with specific such as identification data for anesthesiologists, circulating nurses, etc., as well as other pertinent details, including the type of anesthesia, patient age, gender, comorbidities, other diagnoses, operative reports or discharge summaries. As shown in 924, the user can specify a period after the procedure is complete, where one or more healthcare IT vendor systems, 1600, 1600′, 1600″ are queried for any updated patent data that can be added to the CUER 910. Data updates can be varied, including Clinical Documentation and Provider Notes, Updates on the Device and Implantation Data, Imaging, Pathology, and Test Results, Financial and Billing Data, Medications and Treatments Administered, Regulatory and Compliance Data, Care Coordination and Follow-Up Planning, Operational and Workflow Efficiency Metrics. These additional procedure data sources are reviewed post-procedure for updates, and once the user-defined update period is finalized 927, the CUER is set to “Final,” and it is pushed via 921 to the PHCMD 900.

When the complete UDI encounter record is created, a Global Patient Identification (GPID) is created for each unique patient in the Single Client Database 905; it is then uploaded into the aggregated Patient History of CUERs Master Database (PHCMD) 900. From there, the device identity and associated data will be accessible to the patient and to others as authorized by the patient, e.g., the patient's primary care provider (PCP) or by the providers directly treating the patient during the index or subsequent procedures or on an as-needed basis. The device identity and associated data will be compatible with downloading into HIPAA-compliant EHR systems so that it may be seamlessly added to that patient's record at his primary care provider (PCP) or that of any treating provider granted access. The GPID is used when, after the first patient procedure for which a CUER 910 has been created, the GPID created and associated with the patient will enable future procedure CUERs to be linked into the master patient CUER history collection in the PHCDB 900, FIG. 1. For example, in 931, FIG. 9, multiple CUERs 910 will be associated with the patient based on the GPID.

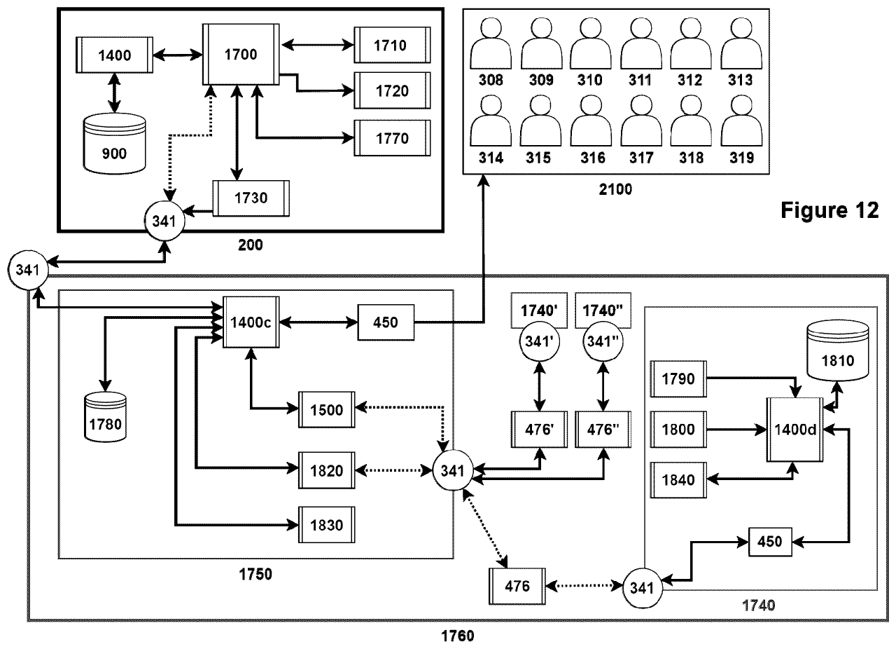

Furthermore, the information in the Patient History of CUERs Master Database PHCMD 900 may be available for queries by third parties for various purposes in accordance with appropriate access rules. For example, the system administrator might assemble a collection of anonymized records for all procedures involving a particular provider, use and inventory data for certain products, or billing data. A manufacturer may access deidentified patient data for a specific medical device. Such anonymized data may be used to examine all records in which the patients had a particular comorbidity (e.g., obesity, diabetes, etc.). Alternatively, regulators, researchers, or payors might access deidentified data to determine safety or effectiveness data for a given device on a population health basis and in specific patient cohorts based on diagnostic or comorbid similarities. The invention portal 1760 and the associated data management services FIG. 12, ensure the data shared with external users 2100 is correctly formatted, users are identified and authenticated, and data is used only according to the usage rights for that user role.

The system further monitors and updates information in the Patient History of CUERs Master Database (PHCMD) 900 going forward. In one example, when a recall notice appears for a particular device, the system might scan all records in the PHCMD 900 and identify any patient who received the device so that the initial healthcare facility, attending physician, the patient's PCP, and/or the patient can be notified. Alternatively, the system may send out an event notification generated from Medical Device Correlator Process 800 that the Event Service Management process 810 issues to all existing Single Client Service Environment users 405, 405′, 405″, etc., which are then processed by performing patient UDI matching in client data base 905 to determine if the event pertains to a known patient 311. The patient's complete UDI encounter record thus becomes a dynamic record, always associated with that patient and available to him, the attending physician, and anyone the patient permits, and the data is not lodged in a silo at the facility where the procedure was done initially.

The following discussion, along with specific examples, will serve to illustrate various aspects of the invention, the system architecture, and various tasks and work products of the associated methods.

### System Architecture.

A central feature of the system architecture is its scalability and ease of implementation. Using techniques to ensure segregation, separation, and encryption at rest and in transit, the patient/medical device encounter data may be provided to end customers in the manner or format they require. It is known that techniques are being developed that will enable having a shared repository with cross-linking (e.g., blockchain). Sharing patient and medical device data sets, according to usage rights, is an actively changing systems research area.

### Example

### Medical Device Database.

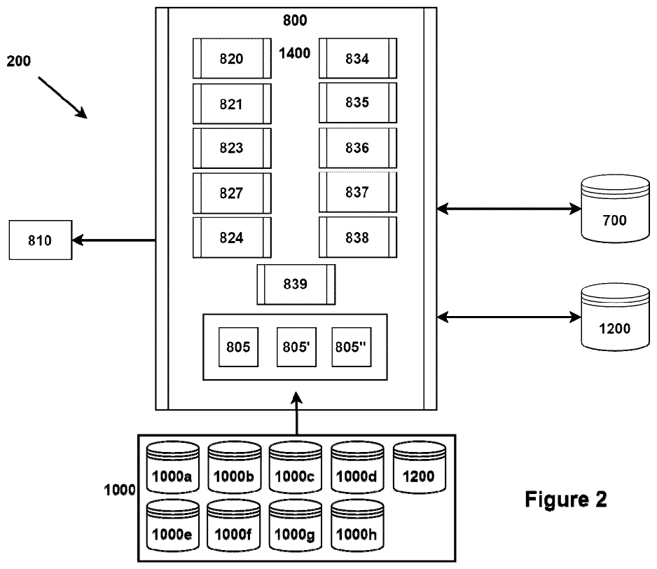

Referring to FIG. 2, in one configuration, the invention maintains a separate, synchronized full copy of the FDA GUDID database, 1000a, containing entries for any regulated medical device active in the marketplace unless exempted by the FDA. The synchronization delay of the local copy with the source copy is a configurable parameter but is set by default to once every 24 hours. The local copy of database 700 is maintained on the central server of the invention, and this central database is queried by the local user web server provided by the invention. The local web server queries the remote MDMD 700 database for each medical device being processed.

The FDA's Global Unique Device Identification Database (GUDID) comprises over 55 data elements for each device record as of January 2024. These elements encompass various categories, including Device Identifier Information, Commercial Distribution Details, Alternative Identifiers, manufacturer Contact Information, FDA Codes and Listing Numbers, Manufacturing Information, Device Dimensions, Storage and Handling Instructions, and Sterilization Information, among other information.

### Example

### Medical Device Recall Database.

The invention in one configuration maintains a separate, synchronized full copy of the FDA Recall database, 1000b containing entries for every recalled medical device. The synchronization delay of the local copy with the source copy is a configurable parameter but is set by default to once every 24 hours. The local copy of the database is maintained on the central server of the invention, and this central database is queried by the local user web server provided by the invention. The local web server queries the database for each medical device being processed.

The FDA's Recall Database comprises over 30 data elements for each recall record as of January 2024. These elements encompass various categories including: Recall Number; Product Description; Reason for Recall; Recall Classification (Class I, II, or III); Recall Initiation Date; Status of the Recall; Recalling Firm's Information; Distribution Pattern; Product Quantity; Event ID; Code Information (e.g., lot numbers, expiration dates); and Voluntary or Mandated Recall Indicator.

### Example

### Medical Device Adverse Events Database.

The invention, in one configuration, maintains a separate, synchronized full copy of the FDA Adverse Events database 1000c, containing entries for every reported Adverse Event that goes back over 30 years. The synchronization delay of the local copy with the source copy is a configurable parameter but is set by default to once every 24 hours. The local copy of the database is maintained on the central server of the invention, and this central database is queried by the local user web server provided by the invention. The local web server queries the database for each medical device being processed.

The FDA's Adverse Event Reporting System (FAERS) and the Manufacturer and User Facility Device Experience (MAUDE) database are key components of the agency's post-market safety surveillance program for pharmaceuticals, biologics, and medical devices. These systems adhere to the ICH E2B (International Council for Harmonization of Technical Requirements for Pharmaceuticals for Human Use—Electronic Common Technical Document) standard for structuring and transmitting adverse event reports. The structured data elements in FAERS and MAUDE encompass multiple categories to ensure comprehensive safety monitoring and regulatory decision-making. The adverse event details contained and leveraged by the invention includes: patient information (i.e., patient age, gender, weight, and medical history to assess the impact of the adverse event across different populations), reporter information (who submitted the report: a healthcare professional, manufacturer, patient, or consumer—to evaluate the credibility and source of the information), suspect product(s) (e.g., the details of the medication or medical device involved, including product name, dosage, frequency, and route of administration for drugs, or Unique Device Identifier (UDI), model number, and manufacturer details for medical devices), adverse event(s) including a detailed description of the adverse event, its clinical outcome, seriousness (e.g., hospitalization, disability, death), and its suspected relationship to the product, concomitant medication lists other drugs or treatments the patient was receiving at the time of the event, allowing the FDA to analyze potential drug interactions or confounding factors, and finally administrative information, includes the source of the report (e.g., spontaneous report, clinical study, literature), the date of submission, country of occurrence, and regulatory tracking details. It is important to note that both FAERS and MAUDE utilize the MedDRA (Medical Dictionary for Regulatory Activities), which organizes event terms into a hierarchical structure, from broad System Organ Classes (SOCs) to specific Lowest Level Terms (LLTs). MedDRA, FAERS, and MAUDE are all included in the invention as data sources 1000 providing ontological and taxonomical enrichment of the FAERS and MAUDE data sources.

### Example

The invention, in one configuration, maintains a separate, synchronized full copy of the FDA Human Cell and Tissue Establishments database 1000d, containing entries for every establishment that works with human cells and tissues classified as medical devices intended for implantation, transplantation, infusion, or transfer into a human recipient. The synchronization delay of the local copy with the source copy is a configurable parameter but is set by default to once every 24 hours. The local copy of the database is maintained on the central server of the invention, and this central database is queried by the local user web server provided by the invention. The local web server queries the database for each medical device being processed.

The FDA's Human Cell and Tissue Establishments Database elements encompass various categories, including Establishment Name: the legal name of the establishment; Establishment Type: which describes the primary function(s) of the establishment (e.g., recovery, processing, storage, distribution, etc.); FEI Number: FDA Establishment Identifier—a unique number assigned by the FDA; Contact Information: address, phone number, email address of the contact person for the establishment; HCT/P Activities: a list of specific activities conducted at the establishment (e.g., tissue recovery, processing, storage, distribution, donor screening, testing); Tissue Types: codes that identify the specific types of human cells and tissues handled by the establishment; Ownership: information about the ownership structure of the establishment (e.g., private, public, non-profit); Accreditation: details about any accreditations held by the establishment related to tissue banking or processing.

### Example

### Master Medical Device Database

The invention may be configured to query the various medical device databases described above or others, many of which are publicly accessible, and some are available under a separate license. However, in one configuration, the databases are consolidated into a master medical device database hosted within the system for ease and speed of access. This master database would be updated on a regular basis by adding whatever new information has been added to any of the constituent databases so that it mirrors the current totality of contents of all the separate databases.

### Example

An essential aspect of the invention is the creation of the medical device master database (MDMD) 700 in FIG. 2. Prior to servicing any system users, MDMD 700 must be created. This process begins with the FDA master device list, called the GUDID or Global Unique Device Identifier Database 1000a. All other databases 1000b to 1000h and their contents are then Correlated 800, using a variety of pattern matching, similar search, and data normalization techniques as are known in the art. Operations include scanning sources and updating master databases 820, matching source items to UDI 821, converting source data to SINDF 827, matching source items in other source databases 823, and resolving item's semantic relationship(s) 824, Ontology or Taxonomy Loading and Parsing 834, Ontology Mapping and Alignment 835, Ontology Reasoning and Inference 836, Ontology Integration and Data Linking 837, Ontology Transformation and Serialization 838, Ontology, MDMD, and PHCMD Enrichment 839. Specific data workflows 805, 805′, . . . represent plug-ins tailored to specific source data types, enabling the system to expand over time with additional data that get harmonized into MDMD 700. The correlator process 800 can also ingest taxonomies and ontologies through the correlation process. Source Taxonomies and Ontologies are available for integration into either the Medical Device Master Database 700 or the Patient History of CUER Database 900. Workflow processes related to Ontologies and Taxonomies include 834, 835, 836, 837, and 838. Ontologies, Taxonomies, and Internal Databases 1000, 700, 1200, and 900 can all be enriched via 839.

FIG. 2 illustrates schematically the data correlation process that unifies the disparate medical device data, ontological, and taxonomy data from a variety of different source databases 1000 into a unique master database 700. This is done in order to reduce the time needed for a medical device's fitness-for-use verification process and other improved efficiencies. Without this, processing each UDI label could potentially take up to 3-5 minutes or more. With this aspect of the invention, search results are returned quickly (in near real-time, based on Applicant's tests).

The following table details some of the types of information managed in the medical device master database 700. All data matching is performed using pointers or links to minimize performance bottlenecks.

TABLE 1
Summary of typical information items that may be managed in the MDMD.

| Name | Function |
|---|---|
| dbo.ADE_Groups | ADE Items that share the same |
| | report_key are given a Group ID |
| | to help track any recent updates |
| | or possible changes |
| dbo.AppVersioning | Contains the current App version |
| | for the mobile app |
| dbo.Device_PI | Table containing the parsed PIs |
| | found in: Recalls, ADEs tables |
| | from the Open FDA Database. |
| | Used for Correlating |
| dbo.Logging | Log files will be created from the |
| | Correlator application. They will |
| | contain errors and updates to existing |
| | items in the MDMD Database. |
| dbo.Medical_Device | The master collection of data obtained |
| | from the FDA GUDID |
| dbo.MedicalDeviceADEAssociation | Medical_Devices with associated ADEs |
| dbo.MedicalDeviceRecallAssociation | Medical Devices with associated Recall |
| dbo.Recall_Groups | Recall Items that share the same |
| | res_event_number are given a Group ID |
| | to help track any recent updates or |
| | possible changes. |
| dbo.UDI_DI_Group | UDIs are given Group IDs to help track |
| | any recent updates or possible changes. |

### Example

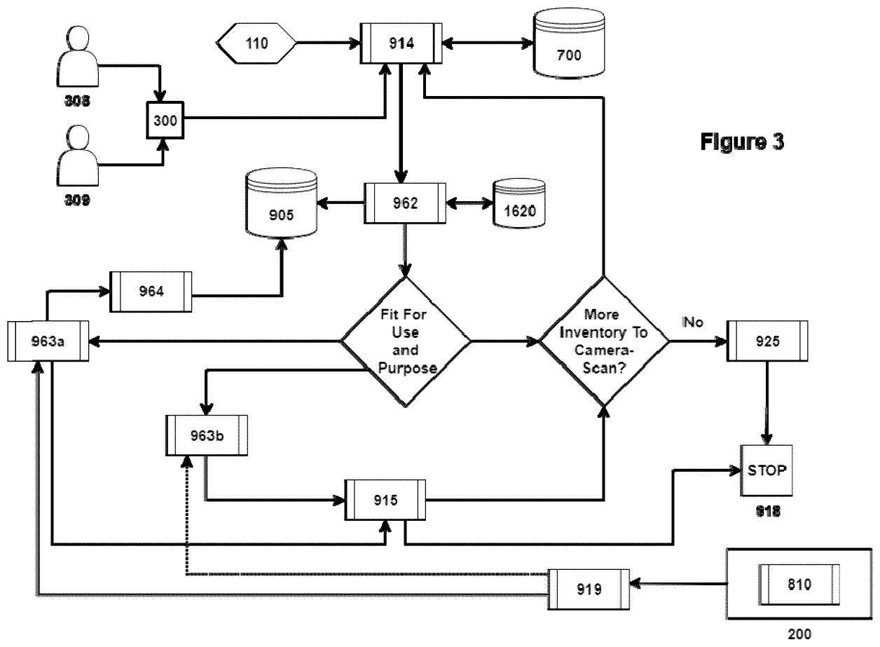

FIGS. 3 and 4 illustrate some other aspects of the Invention and, more particularly, how different classes of users interact with the system in the context of the overall workflow.

FIG. 3 shows a high-level representation of the UDI-centric workflow for devices camera-scanned for validation, specifically in the cases where new medical devices are added to inventory or inspected while unused but in inventory. The steps in FIG. 3 can be performed in receiving or once products have been stored in the inventory's final location. In accordance with some aspects of the invention, a clinical user 309 or a supply chain user 308 using a camera-enabled device 300 capture the UDI label image data, which is then assessed as Fit for use or Not Fit for use 914 (consulting Medical Device Master Database 700). The medical device resulting from 914, whether the device is Fit for Use or Not, is stored in the local client database 905. Next, if the device is not fit for use, it will be due to the product being expired, recalled, or having adverse event reports. Expired and recalled medical device products are immediately deemed unusable. Devices with adverse events can be reviewed and classified as usable or not by the system administrator 313 through the administrator management system, 963a. The items with adverse events are updated based on the administrator's decision on the severity of the adverse event, and database 905 can be updated according to 964. For recalled items, the administrator can process these devices via 963b. In both cases, the final device disposition is handled via 915. Additionally, as some users will want to link their system 905 with third-party software provider 1600, see FIG. 5A, since some users will integrate system 905 with their healthcare IT vendor ERP the system maintains a detailed inventory record in the client database 905; and this step is performed via 962. The medical device correlator 800 will periodically rescan source databases 1000 to determine if there are new UDI entries, new or changed recalls, or new or changed adverse events. When recall or adverse event changes are detected, the correlator 800 will send notifications of the change(s) to all the single client service environment 405 event notification handlers 919. Suppose new adverse events are received that match the inventory on hand. In that case, the administrator can make the appropriate decisions on what to do for the current medical device (DI), and following scans of the same in 963a. If the new recall is received matching inventory on hand, the administrator can make the appropriate changes at 963a. In either case, the devices are finally handled in 915.

FIG. 4 is a schematic diagram showing a user session interface 301 detailing the use of a camera-enabled single device 330, which may be a phone 331, tablet 332, or desktop computer 333 usable by multiple users 308-319 at different times, and the access architecture 320 needed to connect 340 to the client's corresponding Single Client Service Environment 405. A User Session 301 can also be used for medical device validation, as indicated at 914 in FIG. 3.

### Example

The system is configured with a generalized data ingestion and normalization processing, enabling data for broad classes of patient health data to be collected. Data collections include at least the following: 1) inventory management, enterprise resource management, purchasing data, and patient billing; 2) clinical data, patient demographic data, which can be provided as identified or deidentified; 3) data for network-enabled medical devices providing data reporting, exception events, or continuous streaming patient biometric and device status/operation data; and 4) a broad range of consumer and prescription grade biometric monitoring devices.

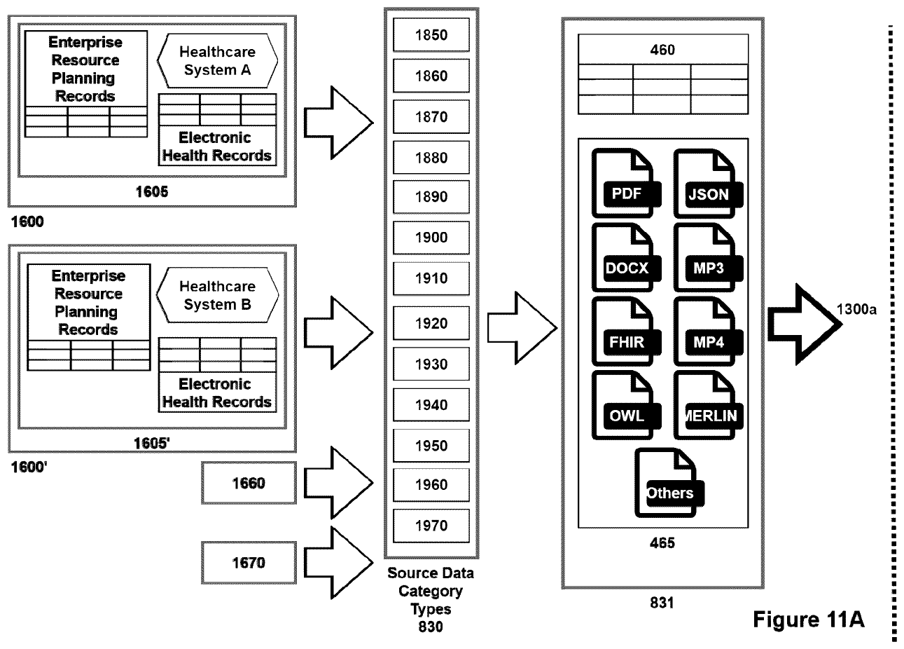

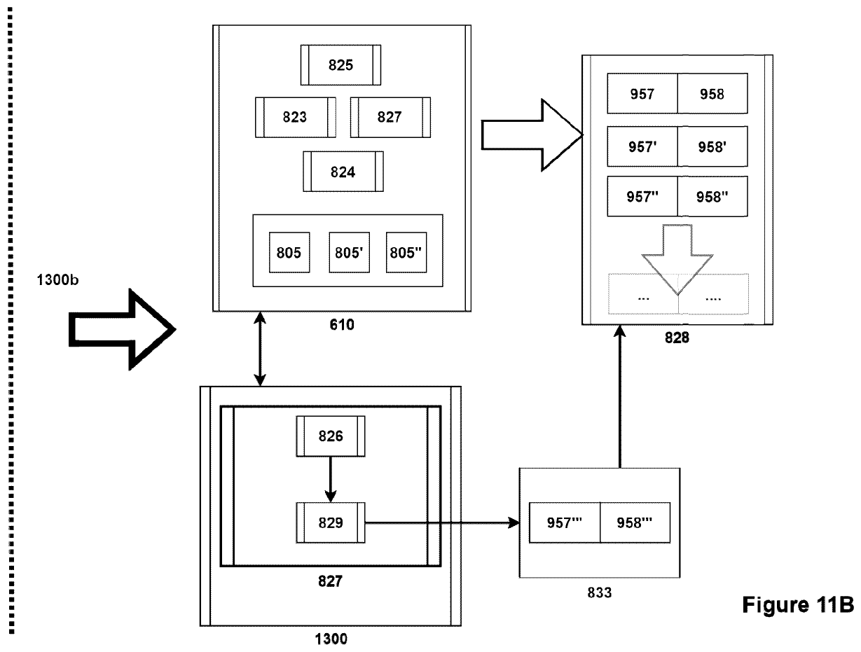

FIGS. 5A-B illustrate the interconnection between the client service environment 405 and a healthcare IT Vendor System 1600 (i.e., a HIT system provided by a third party, e.g., Oracle Health EHR, EPIC, Cerner, athenahealth athenaOne, Medtronic CareLink cardiac device monitoring data, genomic data such as FASTA or BAM data, ontological data such as OWL, or products from vendor such as EverHealth DrChrono, etc.) The connection is preferably made via a Public RESTful API 340. HIT system 1600 has a corresponding store of data 1610. 1610 includes the broadest range of healthcare datasets, as detailed in FIG. 11A, 830 and 831, for example, medical device live network monitoring data, genetic data, ontological data, and so on. In this invention, operationally focused ERP or inventory system datasets 1620, and clinically focused datasets 1625 may be obtained from a patient electronic healthcare records system or from surgical procedure planning and tracking systems. Data inbound from the Healthcare IT Vendor System is made accessible through the connection manager 1500. The connection is defined with the specific data format to be received, and the Standard Class/Element Convertor 1300 puts the data into the appropriate formation. Ingested data can be structured, unstructured, semi-structured, or time-based/longitudinal. Data is formatted in the System Internal Normal Data Format (SINDF) for input into client system correlator process 600. Incorporated into the SINDF is the data format standard United States Core Data for Interoperability (USCDI) and the Observational Medical Outcomes Partnership (OMOP), various Ontologies, Taxonomies, and other information that are related to either a unique medical device 110, a single patient 311, or the domains of medicine, biology, genetics, materials science, device manufacturing, medical device healthcare impacts or influences, and mechanical engineering. All the domains supporting the Medical Device Lifecycle are considered to be included in the invention. This aspect of the invention ensures that the leading emerging standards for data sets will always be mappable into SINDF from various source data formats.

### Example

As shown in FIG. 5A, the Single User Account Client Service Environment (SCSE) 405 is the private, data-protected online cloud infrastructure that services a single user account of the invention. The system creates a separate SCSE for each user organization, e.g., 405′ and 405″. Each client service account 405, 405′, 405″ connects to the patient medical device management environment 200 via a private RESTful API 341. Note that communication links on the right edge of FIG. 5A connects with the left edge of FIG. 5B.

As shown in FIG. 5A, the Client Service Environment captures and manages all user-specific details processed by Invention, including a list of inventory items 917, a collection of records detailing patient procedures 905 and 912, and validation of fitness for use and correctness for the use of medical devices 914. These system services implement all the system's capabilities. System Services 430 provides data processing, transformation, exchange, and maintenance functions, which are associated with and implemented through the Command Execution Management subsystem, 1400b. As shown in FIG. 3, a user on the upper left can access the system by scanning labels of medical device packages, activity 914. These items can be stored and tracked within the invention as individual product items in tables within at 905. Based on the continuous updating of the invention's medical device master database 700 by the correlator FIG. 2, 800, changes in status (e.g., newly recalled, new adverse events, or inventory stock day-to-expiration hits some minimum until it becomes expired) via 810 and 919. When the system is used to document a medical procedure, the Create Encounter system service (912, 920, 921) manages the connections to the HIT vendor systems 1600 for patient clinical and demographic details. The specifics for accessing schedule data and surgeon preference cards are collected using FHIR or other HIT system interoperability standards and tracked for reconciliation at the end of a procedure.

### Example

As shown in FIG. 5A, system services 912-917 depict systems processes and workflows that take place from time to time, based on the current processing stage. Two primary functions of this Invention are to maintain a listing of all medical devices captured for processing within the system. A key feature of the invention is that it can include identifying and tracking inventory using UDI and having the results saved in 905 or optionally pushed externally to 1620. The invention, in its most basic embodiment, is a HIT system peer-level stand-alone platform that eliminates the need for existing inventory or ERP system upgrades to implement UDI. The system can use RESTful APIs to interface with inventory and ERP systems. [REST (Representational State Transfer) is a software architectural style that was created to guide the design and development of the architecture for the World Wide Web.) A second key feature is the creation of the Complete UDI Encounter Record, storing all the medical product identities and clinical, procedural, demographic, provider, and facility data associated with a medical procedure.

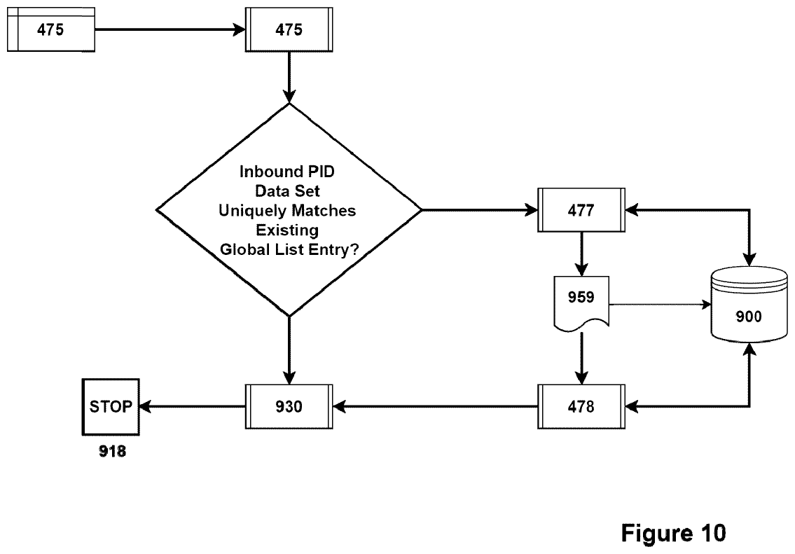

Separately, complete UDI encounter records found within client service environment 405 will have their data aggregated into the Patient History of CUERs Master Database (PHCMD) 900, as shown in FIGS. 1, 5B, 6, 8, 10, and 12. Each of the primary subsystems of the invention 100, including the Patient Medical Device Management Environment 200; one or more of 405, the Single Client Service Environment; and in the portal 1760 Patient History of CUER PHC Portal Environment are supported by a Command Execution Management Subsystem, including 1400c and 1400d. Command Execution Management Subsystem, 1400 in general, is the glue that ties together the data processing routines and workflows for that particular subsystem. These include items such as 1710, 1720, 1770, 1790,1820,1790,1800, and 1840, all within the Portal 1760 and the Patient Medical Device Management Environment 200.

Continuing with the invention design in FIG. 5A, the details of 5B are as follows. Each client service environment 405 connects back into the Patient Medical Device Management Environment 200. The connection manager 1500 keeps track of all the 405 instances attached to it, enabling processing to be performed on a per-client basis. All inbound data, once tagged by the connection manager 1300, is analyzed and processed by the Command Execution Management subsystem, 1400a, which in this figure shows processing for functions 828, 930, 810, and 1700. If the connection contains a stream of data, processing is performed using 1300, 600, and 1200. The processing of 1300 is depicted in FIGS. 11A-B. The processing on the lower right, including 800, 700, 1000, and 1200, is fully depicted in FIG. 2. Finally, the User Portal Execution Management 1700 is depicted in FIG. 12.

### Example

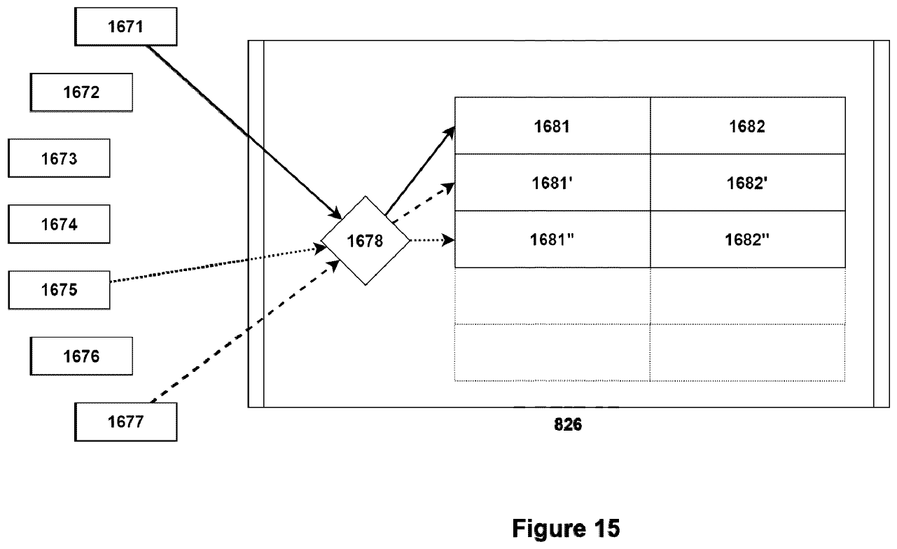

FIG. 15 is an expansion of FIG. 11B showing the conversion process from various medical data formats into the SINDF format 1550. Depicted are the different data sources, each with a 1 to 1 mapping to a conversion template 1682. Healthcare taxonomies, 1677, comprises a general grouping of all healthcare taxonomies, treated as a group but processed individually according to each taxonomy's specific data definitions. It includes the following, among many more: ICD International Classification of Diseases, ICD, a globally recognized system for coding diseases, conditions, and causes of death; used for clinical documentation and billing. Systematized Nomenclature of Medicine—Clinical Terms, SNOMED CT, a comprehensive, hierarchical clinical terminology that standardizes medical concepts for electronic health records and decision support. Logical Observation Identifiers Names and Codes, LOINC, a universal standard for identifying laboratory tests, clinical measurements, and observations in electronic health records. RxNorm, a standardized nomenclature for medications, integrating drug names, ingredients, dosages, and manufacturer information to support interoperability. NDC (National Drug Code, NDC, a unique identifier for medications in the U.S., primarily used for pharmacy transactions, insurance claims, and drug regulation. Current Procedural Terminology, CPT, a set of codes maintained by the American Medical Association (AMA) for billing medical, surgical, and diagnostic procedures. Healthcare Common Procedure Coding System, HCPCS, a coding system used in the U.S. for medical procedures, durable medical equipment, and non-physician services, including Medicare and Medicaid billing. Medical Dictionary for Regulatory Activities, MedRA, a global standard for coding adverse events, drug reactions, and medical device failures, primarily used in pharmacovigilance and regulatory submissions. Observational Medical Outcomes Partnership Common Data Model, OMOP CDM, a standardized framework for integrating real-world healthcare data from EHRs, claims, and registries to enable large-scale observational research.

### Example

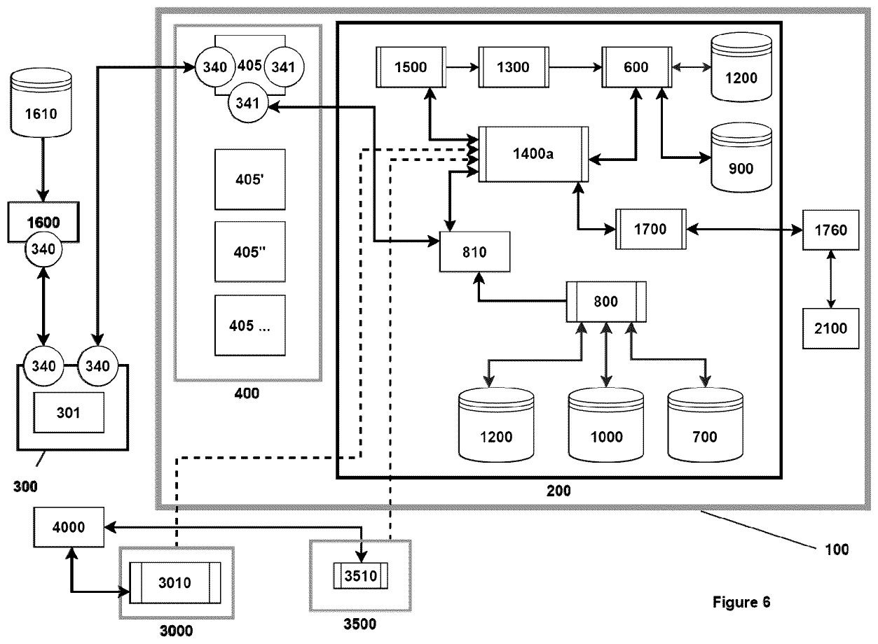

FIG. 6 illustrates the system architecture diagram of the invention 100. Internal to the entire system 100 are the major elements, including, first, 400, the Organization Service Environment Master, which is the management, billing, data sharing, and security envelop that every single client service environment 405′, 405″, .. is contained within. There is no architectural limit on the number of 405 instances that are supported. Next is the Patient Medical Device Management Environment 200, which is the primary data processing engine for the invention. Within 200, the medical device master database 700 is created and maintained up to date by the medical device correlator 800. Events related to changes in recalls, adverse events, or expiration data are sent to users 405 via the event service management function 810. Patient data is ingested and normalized into SINDF format 828 via modules 1300 and 600, using data from 1200 and 900. User access to data via a portal is provided by 1700, 1760, and 2100.

The system server interacts with client devices in several ways:

### Example

FIG. 4 illustrates the several ways in which a user can interact with the system of the present invention. The two primary access modalities are 1) using a camera-enabled device 330 to validate a medical device 914 or 2) performing backend administrative functions 963a and 964, among others. The goal of user interaction design is to enable 323 to be platform-independent. This means the validation software 914 can run on any brand of mobile device operating platform, and administrative functions, including 963a and 964, can run on any internet browser. A dedicated system application, 323, provides access. The system interface is employed by users (308-319) to access the functionality of the system. This term refers to the mobile device (330) application (320) that camera-scans and decodes the UDI data from the physical label, as well as the Administrative Functions. The specific software employed and the features provided are defined by the workflow within the invention. For example, in FIG. 7, software 914, depicts which illustrates the UDI label validation for pre-procedure cart pull workflows; 323 is a mobile application that camera-scans a UDI label from a medical device 110, determines fitness for use, and takes the appropriate actions based on the validation results. In FIG. 3, however, 323 provides for system administrator backend data management related to adverse event handling 963a and 964. This software could run on a mobile camera-enabled device 330 but will more typically be run using a general-purpose computer 333. In either case, the software connects, typically over a public internet connection 340. However, a private network configuration is possible but not depicted. All data is sent to the local client service environment for processing 405.

FIG. 4 shows the core workflow that is then used in any Figure containing 914.

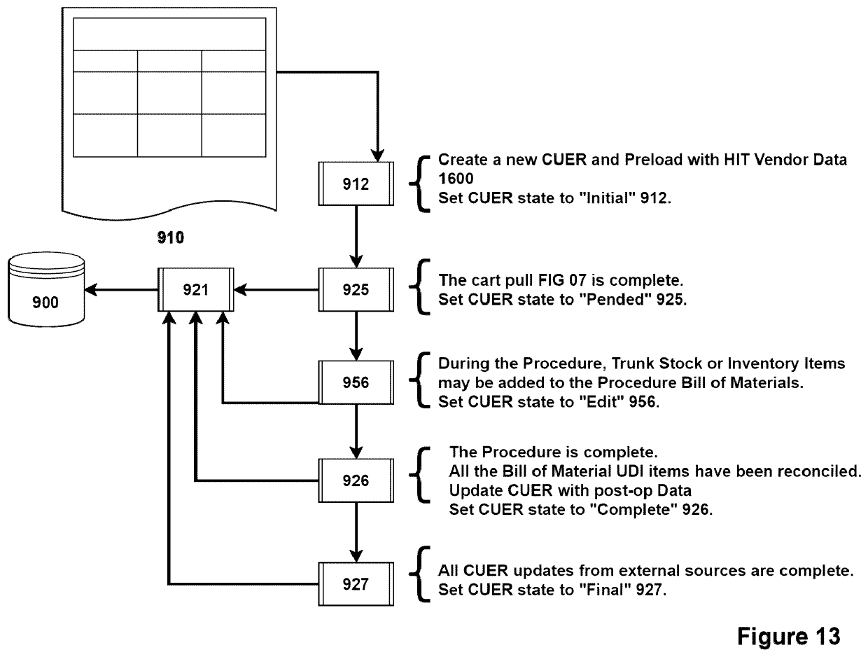

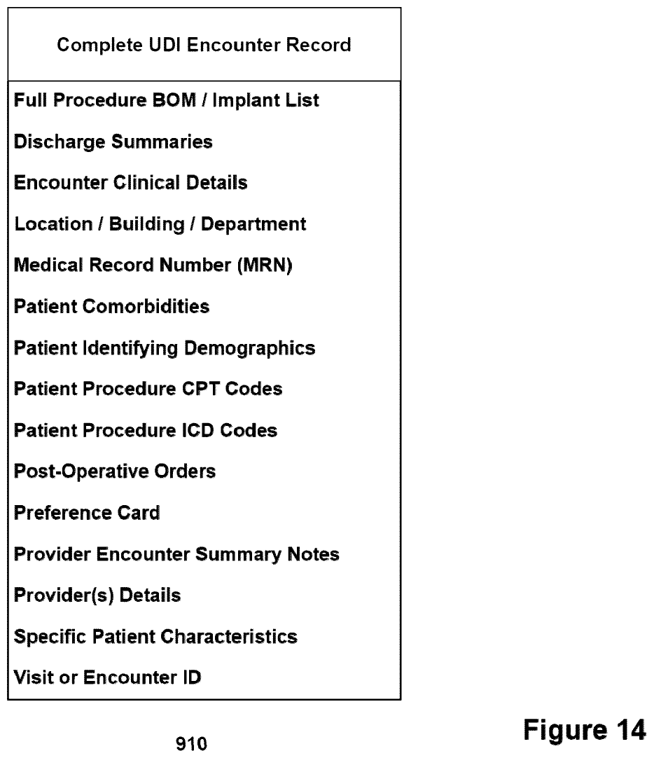

For example, in FIG. 7, a user, 309, will, once a procedure is scheduled in 911, begin the creation of the CUER 910 by initiating step 912. (See FIG. 14 for details of the data types to be included in the CUER.) In 912, the user will select the corresponding software 323 to initiate the creation of a new, patient-specific CUER. The user will identify the patient, schedule, and procedure data, and depending on whether or not there is a direct connection to the appropriate healthcare IT systems 1600, automatically prepopulate the CUER or manually input information on a procedure to be performed, including patient information, medical history, diagnosis, etc. The new CUER 910 may be created during the cart pull procedure FIG. 7, or by some other clinical user 309 prior to the scheduled cart pull. Typically, the steps detailed in FIG. 7 include the following. A new scheduled procedure notification is received from the client EHR based on the encounter created for the patient for the planned procedure or the emergency procedure. This begins the process of building a Complete UDI Encounter Record 910 for the procedure and patient. The initial step in the creation of the final CUER is to auto-populate data fields if there is a direct link to 1600 or manually otherwise. The newly created CUER document with details of the patient, procedure, provider, and medical devices pulled from inventory in preparation for the procedure. Refer to FIG. 13 for details of the CUER creation process and the states the CUER will be undergoing while being “Finalized.” These steps begin with creating the CUER record that is set to be in the “Initial” state 912. Note that the complete UDI portion will be created at the time the procedure is completed. The user will designate the procedure as complete, and all UDI data will be included in the CUER, at which point the status will be set to “Complete” 926. However, the patient and clinical data fields may be finalized at a later time, depending on the circumstances of the encounter. The final CUER will be the complete CUER, which contains all the UDI data for the procedure plus designated additional data elements. Additional data elements will be added to the complete CUER in order to convert it to the final CUER. The client can determine the criteria to define the final CUER. The final CUER is a unique creation of the invention. The final CUER is maintained longitudinally and forms the basis for analytic processes 927, 921, and 900.

Second, another client device may comprise an Automatic Identification and Data Capture (AIDC) system to capture the UDI label information for each device intended for use in a particular procedure. The server assesses Fitness for Use by comparing the acquired UDIs to information from FDA recall and adverse event databases (or, more precisely, to information in the master device database) to determine that the device has not been recalled and compares the use date to the product expiration date to ensure that the product will not be expired as of the use date. The use date is determined by the scheduled date for the procedure, which will already have been auto-populated into the system. When the CUER was first created, the server may further assess Correctness for Use by comparing the device data in the master medical device database with the specific procedure to be performed and the diagnosis of the patient, based on at least one consideration from the group consisting of: laterality; consistency with diagnostic indication; size; patient allergies; patient comorbidities; compatibility with other devices to be used in the surgery; and compatibility with existing implanted devices in the patient. These steps can occur prior to or during the procedure, as noted above.

At the conclusion of the procedure, the system creates a Complete UDI Encounter Record that may contain some of the following or other information contained in the USCDI data set or other data: patient name and demographic data, comorbidity data, patient medical history, if known, patient procedure ICD and CPT codes and other clinical encounter data recorded in the electronic health record system for the patient, identifying information about the physician/provider and the facility in which the procedure was undertaken, and, a complete list of the UDIs for all regulated consumable and implanted devices used in the procedure. Additional data, such as operative reports, discharge summaries, and progress notes, can be added after the conclusion of the procedure to form the final CUER.

Based on user preference, selected data may be sent to the provider's healthcare information system 1625. When the procedure is complete, provider 310 sets the CUER status as “Complete” 926. The complete CUER is then pushed 921 to both the single client database 905 and to the PHCMD 900, which contains records for all clients using the system. For all the devices that were used in the procedure, the provider's third-party ERP or inventory system has been updated 1620. Any devices that are deemed Not Fit for use (expired or recalled) are ultimately processed 929 by disposal, return to the manufacturer, or other means as dictated by client protocols.

The system creates and maintains a Patient History of CUERs Master Database (PHCMD) 900 that includes all Final UDI Encounter Records created as described above. These can be made available to approved users, e.g., the patient, the patient's attending physician, the client facility, the patient's primary care provider (PCP), or others with permission as identified or deidentified records as appropriate. The system may periodically scan the Final CUER to determine if any implanted devices have become the subject of a recall or adverse event. If so, the system may send an alert to the patient, the attending physician, the Client Facility, or the patient's PCP. The system may further make anonymized CUERs of selected patient cohorts available for longitudinal studies.

### Example

Patient History of CUERs Master Database 900 with diverse patient data.

Referring to FIG. 5, every Single User Account Environment 405, 405′, 405″ will generate UDI-based data stored in the Single Client Database 905. Data from each separate client and their respective Single Client Database 905, 905′, 905″ have their CUER data aggregated by the invention. Collecting this healthcare information on behalf of the client organizations and their patients enables the creation of the longitudinal data set containing patient demographics and health status information tied to their implanted medical devices. Information is provided through internetworked API connections 341, including information acquired from at least clinical and ERP systems, as well as patient biometric devices. The invention will map healthcare source data 1610 from any appropriate source Healthcare IT system into a System Internal Normal Data Format (SINDF). The invention maps patient HIT data elements from arbitrary backend Healthcare IT systems into the single, integrated record that is created in the Single User Account CSE 405 within the Single Client Database 905. Once created or updated, the Final CUER is maintained in the Patient History of CUERs Master Database (PHCMD) 900. The final CUERs are indexed to the patient using the GPID, which is created when the CUER is completed. The GPID is created by the invention using patient-specific data elements derived from the Healthcare IT Vendor system 1600. The data collected is used to make the complete UDI encounter record of each procedure for each single patient. Since there are multiple emerging healthcare data format standards, the Standard Class/Element Convertor 1300 in FIG. 11 handles the processing, conversion, and normalization of incoming data based on types (e.g., FHIR, X.25, etc.). The invention reads clinical encounter data from the client healthcare IT system via the Connection Manager 1500 in FIG. 5 and ensures all read data will be formatted and normalized to the System's Internal Normal Data Format.

As shown in FIG. 11, healthcare data can originate from a variety of sources, generating data in at least ten broad categories of source data types. Specific formats in each of these categories can be deterministically mapped to the System Internal Normal Data Format. This aspect enables the patient-medical device data to be stored independently of the originating healthcare systems. It also enables the creation of an active surveillance system for detecting device failures and safety signals. Module 1300 has been spread over FIGS. 11A and 11B for clarity, as indicated by the dashed line between parts 1300a and 1300b.

### Example

### CUER Workflow—Pre-Procedure

Referring to FIG. 7, another aspect of the invention is the creation of a ‘meta-encounter’ collection of data that will memorialize a single patient clinical encounter, including patient data and a complete list of medical devices used in the procedure, including any implanted in the patient. Unlike clinical systems that capture clinical data, or operational systems that capture operational data, the invention creates a master complete UDI encounter record of a patient procedure that will become a record with a complete listing of all of that patient's procedures and stored in the PHCMD 900, and that is inclusive of Patient Information, Device Information, and Procedure Details related to the providers, the facility, clinical notes, and more.

The invention creates and maintains an enriched patient clinical encounter database called the Patient History of CUERs Master Database 900. Existing conventional databases do not have this level of completeness, are not available to those outside the originating system HIT system, do not enable users to carry on long-term device surveillance, and therefore do not offer support for medical care decisions or potential medical device analyses and studies that are made possible by the invention. The Patient History of CUERs Master Database 900 contains real-world evidence tied to specific medical devices and their UDIs and tied to the uses of a serialized medical device by a single identifiable patient. Each encounter record is tied to a unique patient identified in the system by the GPID. That unique patient represents one or more encounter records that detail the data associated with all encounters tracked by the system. The invention creates the complete UDI encounter record, which is stored in the aggregated Patient History of CUERs Master Database 900 and is accessible to various users to facilitate the health care of the patient even for treatments unrelated to the index procedure. For example, the determination of MRI conditionality of an implanted pacemaker would be detailed in the CUER. The system keeps track of all customer accounts using a method of CUER organization based on the identity of each specific patient based on the GPID. Patients are tracked, and their data is accessed independently of their originating healthcare facilities.

### Example

### Correlator Platform Process—Medical Device Master Database

Referring to FIG. 2, the invention relies on medical device correlator process 800, a general-purpose plugin framework for the development and deployment of targeted rules and corresponding data transformation processing operations. The goal is to integrate both structurally and semantically any additional data that is available on a specific medical device or in the encounter history of a patient. The framework will take structured or unstructured information (e.g., clinical charts, device information, biometric readings) and normalize and format the data into the main data standard format. Plugins to the medical device correlator include database-specific scanners for updates 820, matching data items to UDI 821, converting data into SINDF 827, and enrichment-specific routines 823 and 824.

### Example

### Backend Service Structure.

Referring to FIG. 6, creating a patient/device management environment 200 includes 1) the dynamically maintained medical device master database, representing the aggregated, cross-matched textually and semantically enriched Medical Device Master Database 700, 2) the aggregated, normalized, segmented, Patient History of CUERs Master Database 900 that contains the current collection of all patient-specific healthcare data, indexed by the patient's Global Patient ID, and with CUERs associated with the patient record in the PHCMD. Data stored in the PHCMD 900 is made available to users through the Portal Execution Management process 1700, which enables data sharing with providers, patients, researchers, regulators, manufacturers, and others based on their role in the system and allowed activities. The combining of medical device data and patient data into a CUER, which is stored in a dedicated master encounter repository, is done by the invention. This database of CUERs contains information on medical devices. It is fully configured with off-the-shelf FDA data and additional data sources from public and private sources, the detailed facts and details of the procedure that implants the devices, and the effects of those medical devices on a single patient's continuing health status. The system uniquely combines clinical and medical device data to enable advanced analytics and new potential machine learning training set models, and improved patient care by providing the identity of implanted devices with detailed information related to the device. Also, access to the PHCMD can provide recommendations for care based on specific patient parameters to improve patient care.

### Example

### Portal Execution Management.

FIG. 12 illustrates the design of the portal system, enabling users 2100 to access data according to their entitlement rights and privileges. All data that will ultimately become available through the portal 1760 originates from the Patient Medical Device Management Environment 200.

Within 200, the Portal Execution Management System 1700 implements data sharing across portal customer users.

The Portal Execution Management system manages a single portal that enables access to subsets of the patient's complete UDI encounter records and medical record data based on user type and credential access rights. This system maintains security and access protocols for portal users. Data provided will be only the data the user has the right to access and only in the authorized format (e.g., patient-identified, or de-identified).

### Example

### Data Access and Permissions.

Basic access to the patient's record by the patient and his healthcare provider is preferably done as in a typical health care portal. When an encounter record is added to the system or later modified, the patient will be notified and given login credentials and/or a link so that the record may be viewed and downloaded if desired. The record will preferably be available for download as a pdf file, but other formats are possible, depending on the data format (image, text, unstructured, JSON, etc.).

### Example

### Normalize Diverse Healthcare Data Sources

FIGS. 11A-B illustrate the System Internal Normal Data Format (SINDF) workflow, showcasing how disparate healthcare data formats are aggregated, standardized, and stored for unified access and analysis. Data originates from various sources, such as Healthcare Systems A 1600 and B 1600′ which include Enterprise Resource Planning (ERP) Records, Electronic Health Records (EHRs), and Patient Medical Device Biometric Data (if any) 1605. Additionally shown are sources of data from 1660 and 1670, both sources of patient health and medical device performance data. These records are identified and categorized into Source Data Category Types 830, such as Medical Device Data, Ontological and Semantic Formats, and others. Specific Source Data File Types 831, like PDF, FHIR, SNOMED CT, and GraphML, are mapped to their respective categories. The Correlator Platform 610 facilitates data conversion by matching the source data type to a pre-configured mapping plugin 805, using the SINDF Data Converter Run-Time Engine 829 to convert the data into the standardized SINDF format 827. During this process, the system enriches database entries by resolving semantic relationships 824 to ensure ontological alignment. The standardized data is stored as SINDF Formatted Data 828, adhering to a generalized data model that includes Source Data Classes and Elements for relational, unstructured, and multimedia data. Finally, the Standard Class/Element Converter 1300 ensures all elements are normalized to a consistent SINDF schema. This workflow establishes a unified, interoperable data repository, enabling advanced analytics, federated learning, population health studies, and improved treatment decision-making by healthcare providers.

### Example

### Data Curation and Management

FIG. 6 includes all the aspects of the invention. First is the creation, update, and use of the medical device master database 700. This database is created with 800, which is used to reconcile and normalize open sources of healthcare data 1000 into UDI-DI indexed master single source of truth about medical devices. FIG. 6 also illustrates the use of ontology and taxonomy data collections 1200 further to enrich both the medical device master database 700 and the Patient History of CUERs Master Database (PHCMD) 900. The process of collecting massive amounts of data, including medical device data and patient health data, requires workflows and capabilities related to data management, data governance, and data enrichment. These elements may include automated workflows for routine data manipulations or semi-custom implementation of data access and exchange tailored to a specific customer/user's requirements. As shown in FIG. 6, the invention includes selected elements that provide command and control over the operation of the invention when implemented as a service. Included are items 3000, 3010, 3500, 3510, and 4000. In 3500, the system provides for the selection of data access model (private to the client exclusively, shared within a federated configuration, for example). Additional customization includes things such as configuration and management of connections to healthcare IT vendor systems 1600 with customized access to vendor data 1600. Each client's unique requirements and data management customization are contained in 3510. For data that is enriched in the medical device master database 700, 3000 provides the tools and data access necessary to provide quality control, data optimizations, and manual data correction and cross-referencing. The data curation process offers in 3010, specifically tuned tools and management workflows based on the source database 1000.

### Example

There are several ways in which users 308-319 of the data collected by the invention can access the data under appropriate access rules. FIG. 12 illustrates how data is available to end-users based on the type of user (Patient, Provider, Manufacturer, Regulator, etc.). The role-based data transformation layer will enable access in the system, typically as follows. The Patient 311 view includes full PHI access for the patient's records. The Provider 310 view provides access to entire PHI for assigned patients. Only anonymized or de-identified data is available in the Researcher 316 view. In the Device manufacturer's view, only device-specific data with fully de-identified patient data is available. In the Regulator 315 view, data access is configurable to meet regulatory requirements. Finally, for the Parent of the Child Patient, the system enables full access to their child's PHI, equivalent to what the child would have access to in accordance with HIPAA rules. Restricted access ends when the child reaches the age of majority (as per applicable laws).

Based on the user role, the system will tailor the portal's user interface to fit the end-user's use case. For example, a Patient Portal Dashboard will display past procedures, medical devices used, and recommendations. It will also allow patients to export their data in standard formats (e.g., HL7 FHIR, PDF, etc.). The system can provide a communication channel to enable direct communication with providers (e.g., for clarifications). For the Provider Portal Dashboard, the system will enable search and filter to access patient data by name, date, or procedure type. The Provider will also have access to analytics tools that offer essential insights into patient health trends or procedure outcomes. Providers may export data securely for integration with EMR systems. The Researcher Portal Dashboard will include a Cohort Builder to enable querying based on anonymized demographic and clinical parameters and data visualization to provide high-level charts and graphs summarizing cohort results.

The data that is available from the Portal Environment 1760 is extracted from the patient procedure and medical device healthcare data stored in the Patient History of CUERs Database 900. The data in the PHCDB is patient-protected and under the strongest levels of security. Different users of the data include patients accessing their data, providers accessing the data on their patients, researchers seeking cohorts of anonymized patient data, or regulators seeking access to selected data. Even medical device manufacturers can access data related to the performance of their devices. The Portal Environment 1760 enables clients (users) to gain access to a copy of authorized data available within their Single Client Portal Service Environment 1740. This Single Client Portal Service Environment is a separate virtualized account offering data analytics (e.g., any third-party offering such as Tableau, a popular analytics and visualization tool for dashboards and reports that can embed dashboards into the portal, or Microsoft Power BI, a business intelligence tool for creating interactive visualizations that can integrate via embedded Power BI reports, APIs, or Microsoft Azure services). These more classical forms of data analytics can be supplemented with machine learning via ML frameworks or APIs. These mechanisms allow for analyzing data patterns, predictive analytics, or recommendations within the client portal. Examples of ML platforms that can be integrated include TensorFlow/PyTorch, an open-source ML framework for building custom models, Azure Machine Learning, which is Microsoft's cloud-based ML service for building and deploying models, among several others from Google, AWS, Data Robot, and others.

The following examples describe various functions of the inventive system and will serve to illustrate how the system interacts with users, client devices, and external databases.

### Example

Steps taken in preparation for a procedure involving regulated consumable devices:
1. Patient identifying information and medical history, provider identifying information, and procedure date, location and codes are input by a user through a client device and user interface, or this may be automated using standard formatting.
2. Physician prepares a list of devices to be used in the procedure which is stored in the client HIT system.
3. All devices are pulled from inventory at the facility and the UDI and expiration date for each is acquired using a client device and user interface and a record of the device identity is logged to the facility server.
4. System queries the medical device master database for each of the acquired UDIs and provides an alert to the user if any hits are found.
5. System compares expiration dates of all UDIs to the scheduled date of the procedure and provides an alert if any device is expired or nearing expiration.
6. System compares UDI information to procedure information to determine Correctness for Use, which might involve such things as laterality (i.e., if a left hip is being replaced, then a left femoral component must be selected for the procedure).

### Example

Steps taken during a procedure involving regulated consumable devices:
1. If additional devices are needed during the procedure (these may include implantable devices, extra packages of sutures or sterile gowns, etc.), their UDIs are acquired and added to the list of devices used in the procedure.

### Example

Steps taken at the conclusion of a procedure involving regulated consumable devices:
1. Inventory is updated to indicate goods that were consumed.
2. A complete list of devices used (with their UDIs) is prepared.
3. The device list is merged with the available patient identifying information and medical history, provider identifying information, and procedure codes available at the conclusion of the procedure. The information previously supplied by a user and any other additional information, e.g., physician's notes or subsequently created documents, are ingested into the CUER to create the final Complete UDI Encounter Record, which is then added to the Patient History of CUERs Master Database 900 housed on the system server.

### Example

### Third-Party Data Access and Permissions.

For example, a hospital or ASC might have an ongoing subscription, giving it access to all CUERs for procedures performed there. A device maker might periodically purchase a focused search according to some selected parameters. Those search results will be placed on the server, and the subscriber who made the request will receive a notification and login credentials for that particular document or web page. The access portal may provide for download as a pdf or other selected file format.

Additional aspects and applications of the inventive system and methods.

Researchers may use anonymized search results for studies investigating specific diseases or classes of medical device functions or applications seeking Real World Data to support healthcare or medical research.

Medical device manufacturers, regulators, healthcare institutions or organizations, and researchers may leverage the raw Real-World Data and derive Real-World Evidence, providing clinical evidence about the usage of medical devices.

Hospitals and Ambulatory Surgery Center Users may access medical device data to aid in device standardization and effectiveness validation prior to entering into a purchasing agreement.

A medical device engineer may access medical device or patient de-identified data to explore new design concepts, leveraging data detailing medical device design data, news, recall data, etc.

It is expected that the volume of patient data in the invention will increase over time. The dataset will be supported by and used by a wide range of data analysis algorithms, data structures, and data storage formats, enabling security, scalability, redundancy, usability, and performance. Additionally, cohorts of patients or selected subsets of medical device data can be used to train Large Language Models or other AI-based solutions for deriving insights from data.

The system is expected to enable predictive analytics to detect early signals of device failures.

The system is expected to enable patient risk scoring for the probability of post-device implant complications.

The system is expected to enable the physician to determine the best medical device for a patient based on the device's historical outcomes compared to the patient's health status, including any co-morbidities.

The system is expected to enable long-term medical device monitoring for safety and compliance.

The system may enable researchers to identify racial or gender based variabilities in disparities in device performance or patient outcomes.

The system may be integrated with or employ Natural Language Processing (NLP) on Structured or Unstructured Data. This would will enable further insights to be extracted from clinical notes, registry reports, and patient feedback. The combination of NLP and Machine Learning is anticipated to be another useful modality of operation.

The system may be adapted to use traditional and AI-Powered Device Alerts and Failure Detection, potentially providing patients and their providers with real-time alerts when devices show signs of failure.

The system may be adapted to automate regulatory reporting workflows (e.g., FDA, EU MDR, NESTcc). LLMs can be trained to provide automated adverse event reporting (e.g., FAERS, MAUDE submissions).

Patients or permitted health care providers may access the patient's data from prior encounters to identify which implanted devices the patient has. This information will allow for optimal treatment decisions in many clinical scenarios. Examples of this would include planning revision procedures involving an identified device, determining MRI conditionality, determination of recall status of implanted devices or whether adverse events have been associated with the identified device.

The system may allow a client to identify and monitor all items in their inventory that are marked or labeled with a UDI. This includes expiration dates, recall status, location within the facility, and associated vendor information related to costs and contracts. This allows improved inventory management, reducing waste and improper inventory levels of products, charge capture, encounter procedure medical device usage and procedure analysis for cost and efficiency. Using this data allows improved business operations of the client such as understanding per procedure costs related to medical device use, data analytics comparing different health care providers within a facility, reordering of inventory, and identification of alternative products within the supply chain for procurement when standard medical devices are unavailable.

It is anticipated that it will become possible to include UDI data with charge submission to payors. The system will allow for identification of all appropriate charges and allow for submitting these charges, meeting any regulatory requirements requiring this data, improving ability of clients to meet charge audit requests.

The system provides real time information to health care providers during procedures regarding authenticity of medical devices, expiration status, recall status of devices, specific identity of a device with respect to laterality preventing implanting of improper device, among other specific data. This information improves patient safety and reduces risks to the patient and the health care system.

The system may allow healthcare researchers to access deidentified data for comparative research studies to identify which specific device(s) has superior performance, safety metrics, complication profiles, or other metrics based on a specified set of patient characteristics including age, gender, or specific comorbidity profiles in order to recommend specific devices for specific clinical situations and patients.

The system will allow manufacturers to access deidentified data regarding their specific products and the patients the products are used for, with detailed associated patient data to better design and market their products.

TABLE 2
List of callout numerals used in the Figures

| Number | Label |
|---|---|
| 100 | Central Server System |
| 110 | Medical Device(s) |
| 200 | Patient Medical Device Management Environment |
| 300 | User Organization Connected Devices |
| 301 | User Session Interface |
| 308 | User - Supply Chain |
| 309 | User - Clinical |
| 310 | User - Provider |
| 311 | User - Patient |
| 312 | User - Technician |
| 313 | User - Administrator |
| 314 | User - Manufacturer |
| 315 | User - Regulator |
| 316 | User - Researcher |
| 317 | User - Student |
| 318 | User - Parent of Child Patient |
| 319 | User - Other |
| 320 | Access Software |
| 321 | Native Device OS |
| 322 | OS Tech Stack for Single UI Across Disparate |
| | Camera Enabled Device (330) types. |
| 323 | User System Access Application |
| 330 | Camera Enabled Device |
| 340 | Public RESTful API |
| 341 | Private RESTful API |
| 400 | Organization Service Environment Master |
| 405 | Single Client Service Environment |
| 430 | System Services |
| 450 | Client Services Environment Web Server |
| 460 | Structured Data |
| 465 | Non-Structured Data |
| 475 | Source Patient ID Set |
| 476 | VPN Secure Segment |
| 477 | Create New Patient-Specific GPID |
| 478 | Add New Patient CUER Record to Patient History |
| | of CUERs Master Database (PHCMD) |
| 600 | Client-Patient Correlator Process |
| 610 | Correlator Platform |
| 700 | Medical Device Master Database |
| 800 | Medical Device Correlator Process |
| 805 | Correlator Extension Plug-ins |
| 810 | Event Service Management |
| 820 | Scan Sources and Update Master DBs |
| 821 | Match Source Item to UDI |
| 823 | Match Source Item in Other Source Databases |
| 824 | Map Semantic Data Relationship(s) |
| | in MDMD or PHCMD |
| 825 | Determine Data Type of Inbound Data |
| 826 | Select Pre-Configured Source to SINDF |
| | Mapping Plugin (805) to SINDF |
| 827 | Convert Source Data To SNF |
| 828 | SINDF Formatted Data |
| 829 | SINDF Data Converter Run-Time Engine |
| 830 | Source Data Category Types |
| 831 | Source Data Single Data File Types |
| 833 | An SINDF 2-Tuple |
| 834 | Ontology or Taxonomy Loading and Parsing |
| 835 | Ontology Mapping & Alignment |
| 836 | Ontology Reasoning & Inference |
| 837 | Ontology Integration & Data Linking |
| 838 | Ontology Transformation & Serialization |
| 839 | Ontology, MDMD, and PHCMD Enrichment |
| 900 | Patient History of CUERs Master |
| | Database (PHCMD) |
| 905 | Single Client Database |
| 910 | A Single Patient's Complete UDI Encounter |
| | Record (CUER) |
| 911 | Schedule Procedure |
| 912 | Clincal User (309) Creates the Patient- |
| | Specific CUER. CUER State = “Initial” |
| 914 | Validate Medical Device Using UDI |
| 915 | Process Unfit for Use Medical Device |
| 916 | Update Procedure BOM |
| 917 | Process Inventory Item |
| 918 | STOP Process |
| 919 | Process In-bound Event Notification |
| 920 | Get Unique Patient_ID Mapping to PHCMD |
| 921 | Push CUER to Patient History of CUERs |
| | Master Database (PHCMD) |
| 922 | Perform Final Procedure BOM Reconciliation |
| 924 | CUER Update Check for New Data According |
| | to User-Defined Schedule |
| 925 | Update CUER Status = “Pended” |
| 926 | Update CUER Status to “Complete” |
| 927 | Update CUER Status to “Final” |
| 928 | Ingest CUER into Patient History of |
| | CUERs Master Database (PHCMD) |
| 930 | Return the Unique Global Patient ID (GPID) |
| 931 | Single Encounter With Multiple Procedures |
| 951 | Patient Demographic Data |
| 952 | Facility and Provider Data |
| 953 | Patient Clinical Data |
| 954 | Schedule Data |
| 955 | Procedure Cart |
| 956 | Set CUER Status = “Edit” |
| 957 | Source Data Class |
| 958 | Source Data Element |
| 959 | Global Patient Identifier (GPID) |
| 960 | Select a Medical Device for Existing Inventory. |
| 961 | Select a Medical Device from Trunk Stock |
| | provided by a Manufacturer or a Sales Rep. |
| 962 | Update Inventory List in the Single |
| | Client Database |
| 963a | Employ the Adminstration Management |
| | System Interface Workflow (ADEs) |
| 963b | Employ the Adminstration Management |
| | System Interface Workflow (Recalls) |
| 964 | Update UDI DIs in the Inventory List |
| | that are Associated With Adverse |
| | Event Changes 963a. |
| 1000 | Local or Remote Full Copies of External |
| | data sources |
| 1000a | OpenFDA GUDID |
| 1000b | OpenFDA Recalls |
| 1000c | OpenFDA Adverse Events |
| 1000d | OpenFDA HCT |
| 1000e | Clinical Trial Data |
| 1000f | Manufacturer Data |
| 1000g | Clinical Research Data |
| 1000h | Public data sources |
| 1200 | Ontology and Taxonomy Data Collections |
| 1300 | Standard Class/Element Convertor |
| 1500 | Connection Manager |
| 1600 | Healthcare IT Vendor System |
| 1605 | Healthcare IT Vendor Dataset (Generic |
| | Representation) |
| 1610 | Healthcare System Data Set |
| 1620 | ERP/Inventory Healthcare IT Vendor |
| | Data Set |
| 1660 | Medical Device Network or Locally Downloadable |
| | Patient Identifiable Health Data |
| 1670 | Additional Source Data Creators or Databases |
| 1671 | United States Core Data for Interoperability |
| | (USCDI) source data |
| 1672 | Observational Medical Outcomes Partnership |
| | (OMOP) source data |
| 1673 | Clinical Data Interchange Standards |
| | Consortium (CDISC) source data |
| 1674 | Fast Healthcare Interoperability Resources |
| | (FHIR) source data |
| 1675 | Claims Data |
| 1676 | Third-Party Registry Data |
| 1677 | Healthcare Taxonomies |
| 1678 | Source Template Matcher |
| 1681 | Source Data Attribute Record Template |
| 1682 | SINDF Conversion Template |
| 1700 | Portal Execution Management |
| 1710 | Role-Based Access Control (RBAC) Manager |
| 1720 | Data Transformation Layer |
| 1730 | API Gateway Management |
| 1740 | Single Client Portal Service Environment |
| 1750 | Master Portal Service Environment |
| 1760 | Patient History of CUER (PHC) |
| | Portal Environment |
| 1761 | Portal Web Server |
| 1770 | Authentication |
| 1780 | Portal User Accounts |
| 1790 | Analytics Solution Integration |
| 1800 | Machine Learning Platform Integration |
| 1810 | Single Client Portal Database |
| 1820 | Analytics and AI-based Platform |
| | Core Integration |
| 1830 | User Portal Authentication |
| 1840 | Single User Environment Customization |
| 1850 | Audio/Video and Specialized Formats |
| 1860 | Compliance and Security |
| 1870 | Data Analytics and Research |
| 1880 | Data Lakes and Advanced Storage Formats |
| 1890 | General Data Formats |
| 1900 | Genomic Data Formats |
| 1910 | Graph and Network Formats |
| 1920 | Interoperability and Integration Formats |
| 1930 | Medical Device Data |
| 1940 | Ontological and Semantic Formats |
| 1950 | Physiological Data Formats |
| 1960 | Text and Documentation |
| 1970 | Other Formats |
| 2100 | Open (Portal User) |
| 3000 | Central Server System Curation Management |
| 3010 | Curation Process for Each External |
| | Data Source |
| 3500 | Central Server System Client Management |
| 3510 | Client Data Configuration Management |
| 4000 | Business Operations Platform |

### Glossary of Terms

Automatic Identification and Data Capture (AIDC): Automatic Identification and Data Capture (AIDC) is a set of technologies that automatically identifies, collects, and records data from objects, images, sounds, or individuals without direct human involvement. AIDC is commonly used for tracking, identification, and verification processes across various industries, including healthcare, retail, manufacturing, and logistics. AIDC technologies include machine vision processing, barcodes: both 1D barcodes and 2D barcodes, radio frequency identification (RFID), optical character recognition (OCR), biometric identification and magnetic strips and smart cards, near field communication (NFC), and others.

Camera-scan: The workflow associated with validating a medical device for fitness-for-use involves using Device 330 and Access Software 320 to capture and decode the image of a medical device package label and extract and decode 323, the UDI bar code.

Single Client Database data collected on behalf of the client, detailing activities, transactions, audits, and inventory data for use operationally or for patient-procedure reporting. The Single Client Database can be deployed in a single organization or private client cloud account, which can be made sharable according to defined levels of access rights. As shown in FIG. 5A, the Single Client Database resides within the Single User Account, Customer Service Environment 405

Comorbidities: conditions that exist in the patient, which might not be the condition being directly treated by the procedure but which might impact the patient's overall health, such as obesity, diabetes, or smoking history.

Complete UDI Encounter Record (CUER) is the baseline dataset created from a single patient procedure encounter. The Complete UDI Encounter Record (CUER) contains a full list of the UDIs for all products used during a procedural encounter and selected data associated with the patient's clinical procedure, patient, and provider. The CUER may grow or refine in format over time, so it should be considered complete only as it contains all UDI data and extends the purely clinical encounter record to now include such factors as operational performance and efficiency, proactive liability and risk management, and better patient outcomes and safety. Additionally, the CUER will maintain historical information on implants that may have been removed from the marketplace. Elements in the CUER typically include all medical devices used (implanted or not) specific to the procedure; Patient clinical, comorbidities, and demographic data; Provider and facility data; and Provider post-op and even peri-op notes, procedural information such as operative notes, or diagnostic reports to name a few. When combined with the procedure's corresponding MDMD medical device data, a new class of Medical Device Performance Analytics becomes possible.

Correlator Extension: A plug-in to the Correlation Platform 610 execution process. An example is shown in FIG. 2, specific for Medical Device Correlation 800, Correlator Extension Plug-ins 805, 805′, and 805″ extend functionality through new mapping templates, logic, and data models. Conceptually implements a single discrete matching rule or matching rule content. Execution of the rule implies the old matching data is replaced with the new data. Defines one or more preconditions for a match. Rules can be for relational and unstructured data enrichment. This covers both the medical device correlation and the client-patient correlation.

CPT/HCPCS codes: The American Medical Association maintains and annually updates a List of Current Procedural Terminology (CPT)/Healthcare Common Procedure Coding System (HCPCS) Codes (the Code List), which identifies all the procedures and services that may be performed during an encounter.

Data—Structured: A structured data collection can be a relational database table or group of interlinked database tables that capture data related to a specific aspect of the system services provided to the client (300 and 400). Multiple structured data collections are defined to address the diversity of data in the system 100.

Data—Unstructured: Non-Structured data can be “Unstructured Data,” which has no predefined formatting (also called “qualitative data”), “Semi-Structured Data,” which includes XML and JSON data with arbitrary content, and “Longitudinal Data,” which contains data in a time series. Data types include audio, video, 3D, or other forms of media data, metadata, text-based data, mixed text, numeric data, and so on related to a specific aspect of the system services provided to the clients. Multiple structured data collections are defined to address the diversity of data in system 100.

Electronic Health Records (EHR): Digital versions of patients' paper medical records designed to streamline and enhance healthcare through centralized, accessible, and comprehensive patient data management. An encounter is created in the EHR for each episode of care a patient has. EHRs include a wide range of information, such as demographics, medical history, medications, immunization records, lab results, procedural records, radiology reports, and treatment notes and plans, which are updated in real-time to reflect the latest clinical activities and decisions. Unlike simple digital records, EHRs are built to facilitate data sharing among healthcare providers, including specialists, labs, and pharmacies, ensuring that patient information travels seamlessly across care settings. EHR systems support improved clinical decision-making by providing healthcare providers with accurate, up-to-date patient data, reducing the risk of medical errors, and enhancing the continuity of care. EHRs also offer patients access to their health information, promoting patient engagement and self-management. Furthermore, EHRs enable data analytics and population health management, allowing providers to identify trends, track outcomes, and improve preventive care, contributing to higher-quality, safer, and more efficient healthcare delivery.

FDA Adverse Events Database (openFDA): A broader data platform that provides a centralized and structured way to access distinct types of adverse event data across FDA-regulated products. It includes data not only on medical devices (MAUDE data) but also on drugs, biologics, and food-related adverse events. It aggregates data from various FDA databases, including MAUDE (for medical devices), FAERS (FDA Adverse Event Reporting System for drugs), and CAERS (Center for Food Safety and Applied Nutrition's Adverse Event Reporting System).

FDA Human Cell and Tissue Establishment (HCT/P) Database: A vital tool for safeguarding public health in the use of tissue used as a device, drug, and/or biologic product. It serves as a central registry for all facilities involved in handling human cells and tissues, allowing the FDA to track these products from donor to recipient, enforce safety regulations, and monitor compliance. By requiring establishments to register and provide detailed information, the database helps ensure the quality and safety of HCT/Ps, reduces the risk of disease transmission, and facilitates prompt action in case of adverse events. The database promotes transparency and accountability, contributing to public confidence in the safety of human cell and tissue transplantation.

FHIR (Fast Healthcare Interoperability Resources): A healthcare data exchange standard created by Health Level Seven International (HL7) to enhance interoperability across healthcare systems. FHIR organizes data into standardized, modular “resources” (like Patient, Observation, and Medication) that represent specific types of healthcare information. It uses a RESTful API for efficient data exchange and supports JSON and XML formats for compatibility and readability across applications, from EHRs to mobile health apps. FHIR also integrates standardized terminologies like SNOMED CT, LOINC, and ICD-10 for consistency and includes security protocols such as OAuth2 and SMART on FHIR, aligning with regulations like HIPAA to ensure secure, compliant data sharing.

Global Patient ID (GPID): The SINDF data representation of a unique human life identifier. This data collection may consist of one or more data elements that are each patient-identifiable. Each patient is uniquely identifiable based on the collection of data elements. Each patient is assigned a Global Patient ID, and this GPID is the index into the Patient CUER Master Database, uniquely accessing each Patient Life, as embodied by the data set collected for that Patient.

Global Unique Device Identification Database (GUDID): contains key device identification information submitted to the FDA about medical devices that have Unique Device Identifiers (UDI).

HIPAA: The HIPAA Privacy Rule establishes national standards to protect individuals' medical records and other individually identifiable health information (collectively defined as “protected health information”) and applies to health plans, health care clearinghouses, and those health care providers that conduct certain health care transactions electronically. The Rule requires appropriate safeguards to protect the privacy of protected health information and sets limits and conditions on the uses and disclosures that may be made of such information without an individual's authorization. The Rule also gives individuals rights over their protected health information, including the right to examine and obtain a copy of their health records, to direct a covered entity to transmit to a third party an electronic copy of their protected health information in an electronic health record, and to request corrections.

ICD code: An ICD code is a unique alphanumeric code used to classify and categorize diseases, injuries, and other health-related conditions. ICD stands for International Classification of Diseases, a globally recognized diagnostic standard developed and maintained by the World Health Organization (WHO). The codes are used worldwide in healthcare and medical research for health record keeping, billing, and statistical tracking. ICD-10 is the current widely adopted version and includes codes with up to seven characters, allowing for a detailed description of the condition. The latest version, ICD-11, was adopted in 2019, with a January 2022 date for the official start date for usage. Wide-scale adoption is expected during 2025 and beyond.

Longitudinal Patient Study: A longitudinal patient study is a type of observational research study that follows the same group of patients over a prolonged period, often months, years, or even decades. This design allows researchers to collect data on each participant at multiple points in time, providing insights into how a range of factors affect health outcomes, disease progression, or responses to treatment over the long term.

Longitudinal Patient Medical Device Efficacy Study: An observational or interventional study design that evaluates the long-term effectiveness and safety of a medical device in a group of patients over an extended period. This study aims to gather detailed insights on how a specific device performs in real-world, long-term patient care, tracking health outcomes, device reliability, side effects, and overall impact on patient quality of life.

Manufacturer and User Facility Device Experience (MAUDE) database: Reports received by the FDA of adverse events involving medical devices. The data consists of voluntary reports from June 1993, user facility reports since 1991, distributor reports since 1993, and manufacturer reports since August 1996.

Medical Device Correlator: A central engine that links UDIs medical devices, patient histories, and inventory data to provide real-time, cohesive tracking and management within the invention. It associates devices with specific patients and procedures, validates Unique Device Identifiers (UDIs), tracks device locations, and generates alerts for maintenance or usage mismatches. By aggregating and analyzing usage patterns, the correlator supports regulatory compliance, optimizes inventory, and enhances patient safety, offering actionable insights that improve operational efficiency and clinical workflows.

Medical Device Recalls: Contains medical device recalls classified since November 2002. Since January 2017, it may also include correction or removal actions initiated by a firm prior to review by the FDA. The status is updated if the FDA identifies a violation and classifies the action as a recall and again when the recall is terminated. FDA recall classification may occur after the firm recalling the medical device product conducts and communicates with its customers about the recall. Therefore, the recall information posting date (“create date”) indicates the date the FDA classified the recall, and it does not necessarily mean that the recall is new

Ontology. A formalized data modeling framework that structures and organizes a domain-specific knowledge base. In this invention, Ontologies focused on healthcare, including Disease Ontology (DO), Basic Formal Ontology (BFO), Foundational Model of Anatomy (FMA), and Gene Ontology (GO), are just a few of the potential ontologies integrated into the invention. These ontologies define a comprehensive collection of concepts, their semantic relationships, attributes, and associated constraints within the specific field. It enables a machine-readable, logically consistent representation of data that facilitates interoperability, reasoning, and knowledge inference across disparate systems. Unlike taxonomies, ontologies go beyond hierarchical categorization by defining complex semantic relationships, attributes, and constraints between concepts within a domain. Ontologies establish formalized rules that describe how concepts relate to each other, allowing for context-aware data integration, automated decision support, and knowledge discovery. This deeper level of semantic understanding is critical in domains like healthcare.

Patient Medical History: A collection of all essential information gathered during the provider visit, including: 1) Patient Medical History: Details about past illnesses, surgeries, allergies, and social and family histories. 2) Physical Exam Findings: Initial examination results, vital signs, and any observed physical abnormalities. 3) Laboratory Results: Data from initial diagnostic tests, such as blood tests, imaging, or other relevant diagnostics. 4) Problem List: Each patient problem is listed here with a unique identifier. The list is continuously updated as new problems are identified or resolved. Problems can be either medical diagnoses (e.g., diabetes), symptoms (e.g., chest pain), or psychosocial issues (e.g., family stressors impacting health). This Patient's Medical History makes it easier for providers to track all active and resolved issues in a patient's care plan.

Primary Care Provider (PCP): A is a healthcare professional who serves as the first point of contact for patients within the healthcare system. PCPs offer a broad range of services, including preventive care, diagnosis and treatment of common illnesses, management of chronic conditions, and guidance on overall health and wellness. They play a significant role in coordinating care across other healthcare specialists and services, ensuring that patients receive comprehensive, continuous, and integrated care.

Procedure: An event at a hospital, an ambulatory surgery center, a diagnostic center, a cardiac center, or other treatment facilities that involves a patient undergoing medical intervention for a variety of reasons. These could include surgeries, therapeutic injections, cardiac stenting, dialysis, or many other types of procedures.

Regulated Consumable Device: A product intended for general consumer use that has a specific medical or health-related purpose and is subject to regulatory oversight to ensure safety, efficacy, and compliance with health standards. Regulatory authorities, such as the U.S. Food and Drug Administration (FDA) or the European Medicines Agency (EMA), may oversee these devices based on the risk they pose to users or the claims made about their health benefits. Examples include disposable products used in procedures, such as sterile drapes, sutures, needles, dressings, and sterile barriers. These devices can be associated with the patient's CUER history either by UDI if regulated or by association with the Global Patient ID, which matches the specific patient.

Regulated Medical Device: Any instrument, apparatus, implement, machine, software, implant, reagent, or similar article that is intended for use in the diagnosis, treatment, prevention, or monitoring of disease or the maintenance of health. These devices are subject to oversight by government agencies, such as the U.S. Food and Drug Administration (FDA), the European Medicines Agency (EMA), or other regional health authorities, to ensure they meet specific safety, effectiveness, and quality standards. It needs to be included in the GUDID and not exempt.

Regulator or regulatory authority: A government body responsible for creating, implementing, and enforcing rules and standards to protect public health, safety, welfare, and the environment within a specific area or industry. Regulatory agencies oversee compliance with laws and regulations, often conducting inspections, approving products, monitoring industry practices, and imposing penalties for non-compliance. They play a critical role in ensuring that companies and individuals meet legal standards to maintain public trust and safety. Activities include rulemaking, licensing and approvals, enforcement, monitoring and surveillance, guidance and education, and public health and safety.

System Internal Normal Data Format (SINDF): A structured, standardized, and flexible format designed to aggregate, digitize, and manage diverse healthcare data elements within a unified system. Its primary function is to store, organize, and facilitate the retrieval and analysis of healthcare information, including clinical history, patient demographics, and device-specific details. The data schema and storage approach for the SINDF collection is a hybrid, scalable, and interoperable system designed to accommodate diverse healthcare data types, including structured, semi-structured, and unstructured data. This storage solution combines the principles of modern database architecture and advanced analytics capabilities to support the needs of healthcare providers, patients, and researchers.

Specialist Provider: A Specialist Provider or Specialist Surgeon is a healthcare professional who has advanced training and expertise in a specific area of medicine or surgery. Unlike primary care providers, who offer general medical care, specialists focus on particular body systems, diseases, or types of treatment. They are often consulted for more complex or specialized health issues that require targeted diagnostic, therapeutic, or surgical intervention.

Source Patient ID Set: A JSON array containing the specific patient attributes provided from the single client environment and used to identify a unique patient on the FHC Master Server. E.g. [{“First”: “Joe”, “SS”: “321234543” }]

Taxonomy: A hierarchical classification framework that systematically organizes domain-specific concepts into categories and subcategories based on predefined relationships. In this invention, taxonomies relevant to healthcare, such as the Systematized Nomenclature of Medicine (SNOMED CT), Medical Subject Headings (MeSH), and the Universal Medical Device Nomenclature System (UMDNS), serve as structured classification systems that enable standardized terminology and categorization of medical concepts, procedures, and devices. A taxonomy provides a structured yet flexible way to classify information, ensuring consistent labeling, retrieval, and analysis of domain-specific data while facilitating interoperability between different healthcare systems. Unlike ontologies, taxonomies primarily focus on hierarchical categorization rather than complex semantic relationships and constraints.

User Organization: An organizational entity and the individuals at the organization that have access to the invention. A User Organization may also be referred to as a Client, a Client Organization, a User or Users.

Unique Device Identifier (UDI): A standardized, globally unique identifier for medical devices. Its purpose is to improve patient safety, enhance traceability, streamline recall processes, and support effective post-market surveillance of medical devices. The U.S. FDA regulates the UDI systems and follows international standards to ensure uniformity across healthcare markets. The regulation defines the UDI Device Identifier (DI), a static, mandatory portion of the UDI that identifies the specific version or model of a medical device and is assigned by the labeler or manufacturer. The regulation also defines the Production Identifier (PI), which is a variable portion that provides additional details such as Lot or batch number, Serial number, Expiration date, and Manufacturing date.

UDI Event Notification: Processes in which the medical device correlator 800 will periodically rescan source databases 1000 to determine if there are new UDI entries, new or changed recalls, or new or changed adverse events. When recall or adverse event changes are detected, the correlator 800 will send notifications of the change(s) to all the single client service environment 405 event notification handlers 919. If, e.g., new adverse events are received and match the inventory on hand, then the administrator can make the appropriate decisions on what to do for the current medical device (DI), and following scans of the same at 963a. If the new recall is received that matches inventory on hand, the administrator can make the appropriate changes at 963a. In either case, the devices are finally handled in 915.

We claim:

1. A method for identifying and tracking medical devices comprising the steps of:
a) acquiring UDI(s) for selected medical device(s) to be used in a procedure on a selected patient;
b) validating each selected device for use by checking at least one device database;
c) performing the procedure on the patient;
d) preparing a file to include specific details regarding the patient and specific details of the clinical encounter, including an inventory by UDI of all medical devices used for the procedure or implanted in the patient with specific device information including fitness and correctness for use for each device;
e) uploading the file to a database of complete UDI clinical encounter records;
f) maintaining the file such that it can be updated automatically or by selected users with information available at a future time and create and send user notifications based on future updates to at least one user;
g) making the file available in a format selected for selected parties under selected access rules.

2. The method of claim 1 wherein said UDI(s) of the medical device(s) are acquired by a device selected from the group consisting of handheld barcode readers, stationary barcode readers, smartphone cameras, tablet computer cameras, and Automatic Identification and Data Capture (AIDC) systems.

3. The method of claim 1 wherein said at least one device database is selected from the group consisting of: the FDA medical device recalls database, the openFDA Adverse Events database; the FDA Human Cell and Tissue Establishment (HCT/P) database; the Global Unique Device Identification Database (GUDID); and an internal database containing information synchronized with any or all of these or other databases.

4. The method of claim 1 wherein said file containing details of the clinical encounter includes information selected from the group consisting of: patient identification and demographics; patient health history and diagnoses; encounter details; procedural details; provider information; attending physician information; patient primary care provider; patient comorbidities; and a list of all devices selected for use along with their respective UDIs and data characterizing each device derived from at least one database maintained by the FDA or other national, regional, or international (e.g. EU) regulatory body focused on medical device identification, and available via public data access.

5. The method of claim 1 wherein said step of adding an individual clinical encounter to said clinical encounter database allows said clinical encounter document to be searchable using the fields of said clinical encounter database.

6. The method of claim 1 wherein an individual clinical encounter record may be updated with information selected from the group consisting of: reported device related adverse events of any device used in said procedure; recall of any device used in said procedure; and revision or removal of said device or related to said procedure.

7. The method of claim 1 wherein said file is made available to at least one user selected from the group consisting of:
said patient;
said patient's primary care provider; and
said attending physician;
said healthcare facility where encounter occurred.

8. The method of claim 1 wherein selected de-identified information from said file is made available to at least one user selected from the group consisting of:
employees of a governmental regulatory agency;
manufacturers of medical devices;
medical researchers; and
other interested parties.

9. The method of claim 1 wherein said file is provided to an external HIT system in a format compatible with said external HIT system.

10. The method of claim 1 wherein said file format is selected from the group consisting of: PDF files; XML files, and XLS files.

11. The method of claim 1 wherein step (d) further comprises converting the contents of said file into a System Internal Normal Data Format.

12. The system of claim 1 wherein step (g) further comprises the step of converting said file from the System Internal Normal Data Format to another selected format for viewing, downloading, printing, data analytics, and machine learning purposes.

13. A method for identifying and tracking medical devices comprising the steps of:
a) providing a server device including searchable databases comprising:
a first database listing commercially available medical devices, with their corresponding UDIs;
a second database listing recalled medical devices and their corresponding UDIs;
a third database listing reported adverse events associated with medical devices and their corresponding UDIs;
a fourth database listing commercially available tissue and biologic devices; and,
a fifth database listing encounter records for medical procedures in which medical devices were used in individual patients, and the corresponding UDIs of said devices;
b) providing a plurality of client devices through which users may query said searchable databases and may upload complete UDI encounter records to said encounter records database;
c) prior to performing a medical procedure, using a client device to:
acquire the UDI for each device selected for possible use in said medical procedure;
query said first, second, third, and fourth databases and reject any selected device that is expired or subject to recall or adverse event or incorrect for the patient; and,
prepare a complete bill of materials for said procedure;
d) after said medical procedure is completed, using a client device to upload an encounter record to said complete UDI encounter record database, said encounter record comprising at least:
date of procedure;
procedure information;
patient identifying information;
provider identifying information;
physician identifying information; and,
complete list of all medical devices used and their UDIs;
Identifying each unique human life using data contained in said encounter record; and,
e) making said complete UDI encounter database accessible at future times to selected users under selected access rules;
f) Intermittently updating the said complete UDI encounter database with information from the first, second, third or fourth databases that relates to the device identified in the complete UDI encounter record, and generating notifications to the selected users based on the updated information.
